(19) United States
(12) Patent Application Publication
(10) Pub. No.: US 2022/0333172 A1
(43) Pub. Date: Oct. 20, 2022

(54) LIVE CELL IMAGING OF NON-REPETITIVE GENOMIC LOCI

(71) Applicant: The Jackson Laboratory, Bar Harbor, ME (US)

(72) Inventors: Albert Cheng, Bar Harbor, ME (US); Patricia Clow, Bar Harbor, ME (US)

(73) Assignee: The Jackson Laboratory, Bar Harbor, ME (US)

(21) Appl. No.: 17/635,424

(22) PCT Filed: Aug. 13, 2020
(86) PCT No.: PCT/US2020/046076

§ 371 Date: Feb. 15, 2022

Related U.S. Application Data
(60) Provisional application No. 62/984,466, filed on Mar. 3, 2020.
(60) Provisional application No. 62/887,913, filed on Aug. 16, 2019.

(51) Int. Cl. C12Q 1/6841; C12N 9/22

(52) U.S. Cl. CPC C12Q 1/6841; C12N 9/22; C12Q 1/6816

(57) ABSTRACT

Provided herein are methods of imaging non-repetitive genomic loci using unique guide ribonucleic acids (gRNAs), an RNA-guided nuclease, and a detectable conjugate.

### RELATED APPLICATIONS

This application claims the benefit under 35 U.S.C. § 119(e) of U.S. provisional application Ser. No. 62/887,913, filed Aug. 16, 2019, and of U.S. provisional application Ser. No. 62/984,466, filed Mar. 3, 2020, each of which is herein incorporated by reference in its entirety.

### GOVERNMENT LICENSE RIGHTS

This invention was made with government support under grant number P30CA034196 awarded by National Cancer Institute and grant number R01-HG009900 awarded by National Human Genome Research Institute. The government has certain rights in the invention.

### BACKGROUND

Two meters of genomic DNA are condensed into the approximately 10 micrometer diameter human nucleus (1). The three-dimensional organization of the genome influences functions such as transcription activity and regulation, DNA replication, and DNA repair (2). Disruption of this structure and these processes has been implicated in disease (3, 4). DNA sequencing approaches including Hi-C and ChIA-PET techniques (5-7) have revealed chromatin interactions within the genome as well as interactions between the genome and regulatory elements, but these techniques require fixation of the chromatin, isolation from the nuclear environment, and fragmentation.

### SUMMARY

Provided herein, in some aspects, are methods for imaging dynamic nuclear architecture and processes within live cells. Live cell imaging of nonrepetitive sequences with CRISPR and TALE, for example, have been hampered by laborious protocols and low signal-to-noise ratios (SNRs), requiring transfection of tens of plasmids to achieve labeling of each locus. The present disclosure provides a CRISPR/Casilio-based imaging method with enhanced SNR, which enables labeling of one nonrepetitive genomic locus using only a single gRNA. This approach can be used to analyze 3D chromatin interactions in real time.

In some aspects, the present disclosure provides methods comprising (a) imaging a live cell that comprises a catalytically-inactive ribonucleic acid (RNA)-guided nuclease, a non-repetitive genomic locus bound by a single unique guide RNA (gRNA), and a detectable molecule linked to a PUF domain that binds to the PUF domain-binding sequence of the gRNA, wherein the gRNA comprises (i) a deoxyribonucleic (DNA)-targeting sequence that is complementary to the non-repetitive genomic locus, (ii) a RNA-guided nuclease-binding sequence, and (iii) a Pumilio-FBF (PUF) domain-binding sequence, and (b) detecting in the live cell the detectable molecule of the PUF domain bound to the PUF domain-binding sequence of the gRNA.

Other aspects of the present disclosure provide methods comprising (a) imaging a live cell that comprises a catalytically-inactive RNA-guided nuclease, multiple non-repetitive genomic loci, and a detectable molecule linked to a PUF domain that binds to the PUF domain-binding sequence of the gRNA, wherein each non-repetitive locus is bound by a single unique gRNA, wherein the gRNA comprises (i) a DNA-targeting sequence that is complementary to one of the non-repetitive genomic loci, (ii) a RNA-guided nuclease-binding sequence, and (iii) a PUF domain-binding sequence, and (b) co-detecting in the live cell at the multiple non-repetitive genomic loci the detectable molecule of the PUF domain bound to the PUF domain-binding sequence of the gRNAs.

Still other aspects of the present disclosure provide methods comprising (a) contacting a live cell with a catalytically-inactive RNA-guided nuclease or a polynucleotide encoding a RNA-guided nuclease, multiple gRNAs, a polynucleotide encoding multiple gRNAs, or multiple polynucleotides encoding a gRNA, and a fluorescent protein linked to a PUF domain or a polynucleotide encoding fluorescent protein linked to a PUF domain that binds to the PUF domain-binding sequence of each of the gRNAs, wherein each of the gRNAs comprises (i) a deoxyribonucleic (DNA)-targeting sequence that is complementary to a single non-repetitive genomic locus in the live cell, (ii) a RNA-guided nuclease-binding sequence, and (iii) a PUF domain-binding sequence, and (b) co-detecting in the live cell the fluorescent protein linked to a PUF domain bound to the PUF domain-binding sequence of the gRNAs.

Yet other aspects of the present disclosure provide methods comprising (a) imaging multiple non-repetitive genomic loci in a live cell, wherein each non-repetitive genomic locus is bound by a single unique gRNA, and a detectable molecule linked to a RBP domain that binds to the RBP domain-binding sequence of the gRNA, wherein the gRNA comprises (i) a DNA-targeting sequence that is complementary to the non-repetitive genomic locus, (ii) a RNA-guided nuclease-binding sequence, and (iii) a RNA-binding protein (RBP) domain-binding sequence, and (b) detecting in the live cell the detectable molecule of the RBP domain bound to the RBP domain-binding sequence of the gRNA.

Further aspects of the present disclosure provide methods for imaging chromatin architecture, comprising: labeling in a live cell a first non-repetitive chromatin anchor locus with (a) a single unique guide RNA (gRNA), wherein the gRNA comprises (i) a deoxyribonucleic (DNA)-targeting sequence that is complementary to the non-repetitive genomic locus, (ii) a RNA-guided nuclease-binding sequence, and (iii) a Pumilio-FBF (PUF) domain-binding sequence, and (b) a detectable molecule linked to a PUF domain that binds to the PUF domain-binding sequence of the gRNA; labeling in the live cell multiple additional non-repetitive chromatin loci, each loci labeled with (a) a single unique gRNA, wherein the gRNA comprises (i) a DNA-targeting sequence that is complementary to the non-repetitive genomic locus, (ii) a RNA-guided nuclease-binding sequence, and (iii) a PUF domain-binding sequence, and (b) a detectable molecule linked to a PUF domain that binds to the PUF domain-binding sequence of the gRNA, wherein the multiple additional non-repetitive loci are located at increasing distances from the anchor locus; and imaging in the live cell over a period of time the detectable molecules, thereby imaging chromatin architecture in the live cell.

In some embodiments, the distance between at least two of the non-repetitive genomic loci is 1 kb to 5 kb, 1 kb to 100 kb, 10 kb to 100 kb. In some embodiments, the distance between at least two of the non-repetitive genomic loci is at least 1 kb, at least 5 kb, at least 10 kb, or at least 20 kb.

In some embodiments, the methods comprise time-lapse imaging of a live cell.

In some embodiments, a detectable molecule is a fluorescent protein.

In some embodiments, a live cell is contacted with at least two PUF domains, each linked to a different detectable molecule. The detectable molecules, in some embodiments, are fluorescent proteins with different emission wavelengths relative to each other.

In some embodiments, a live cell comprises at least two gRNAs, wherein each of the gRNAs comprises (i) a DNA-targeting sequence that is complementary to only a single non-repetitive genomic locus in the live cell, (ii) a RNA-guided nuclease-binding sequence, and (iii) a PUF domain-binding sequence. For example, a live cell may comprise at least five gRNAs, wherein each of the gRNAs comprises (i) a DNA-targeting sequence that is complementary to only a single non-repetitive genomic locus in the live cell, (ii) a RNA-guided nuclease-binding sequence, and (iii) a PUF domain-binding sequence.

In some embodiments, a live cell comprises at least three gRNAs, wherein each of the gRNAs comprises (i) a DNA-targeting sequence that is complementary to only a single non-repetitive genomic locus in the live cell, (ii) a RNA-guided nuclease-binding sequence, and (iii) a PUF domain-binding sequence. For example, a live cell may comprise at least five gRNAs, wherein each of the gRNAs comprises (i) a DNA-targeting sequence that is complementary to only a single non-repetitive genomic locus in the live cell, (ii) a RNA-guided nuclease-binding sequence, and (iii) a PUF domain-binding sequence.

In some embodiments, a live cell does not include a pool of gRNAs.

In some embodiments, the catalytically-inactive RNA-guided nuclease is a dCas9 nuclease.

In some embodiments, at least one of the gRNAs comprises at least one copy of the PUF domain-binding sequence.

In some embodiments, non-repetitive genomic loci or locus comprise(s) chromatin.

Other aspects of the present disclosure provide an in vitro composition comprising a live cell that comprises a catalytically-inactive RNA-guided nuclease, multiple non-repetitive genomic loci, wherein each non-repetitive locus is bound by a single unique gRNA, wherein the gRNA comprises (i) a deoxyribonucleic (DNA)-targeting sequence that is complementary to one of the non-repetitive genomic loci, (ii) a RNA-guided nuclease-binding sequence, and (iii) a PUF domain-binding sequence, and a detectable molecule linked to a PUF domain that binds to the PUF domain-binding sequence of the gRNA.

Some aspects of the present disclosure provide methods for detecting a chromosomal rearrangement in a cell, comprising delivering to a live cell (a) a catalytically-inactive RNA-guided nuclease, (b) a first single unique gRNA that comprises a DNA-targeting sequence that is designed to bind adjacent to and upstream from a nuclease cleavage site, (c) a detectable molecule linked to a PUF domain that binds to the PUF domain-binding sequence of the first gRNA, (d) a second single unique gRNA that comprises a DNA-targeting sequence that is designed to bind adjacent to and downstream from a nuclease cleavage site, and (e) a detectable molecule linked to a PUF domain that binds to the PUF domain-binding sequence of the second gRNA, wherein each gRNA further comprises a RNA-guided nuclease-binding sequence and a PUF domain-binding sequence; and imaging in the live cell the distance between the first gRNA and the second gRNA to determine the presence or absence of a chromosomal rearrangement. In some embodiments, the chromosomal rearrangement is a translocation, an inversion, or a duplication.

Other aspects of the present disclosure provide methods for identifying a genetic abnormality in a cell, comprising delivering to a live cell (a) a catalytically-inactive RNA-guided nuclease, (b) a first single unique gRNA that comprises a DNA-targeting sequence that is designed to bind adjacent to and upstream from a genetic abnormality, (c) a detectable molecule linked to a PUF domain that binds to the PUF domain-binding sequence of the first gRNA, (d) a second single unique gRNA that comprises a DNA-targeting sequence that is designed to bind adjacent to and downstream from a genetic abnormality, and (e) a detectable molecule linked to a PUF domain that binds to the PUF domain-binding sequence of the second gRNA, wherein each gRNA further comprises a RNA-guided nuclease-binding sequence and a PUF domain-binding sequence; and imaging in the live cell the distance between the first gRNA and the second gRNA to determine the presence or absence of a chromosomal rearrangement. In some embodiments, the genetic abnormality is a chromosomal rearrangement. In some embodiments, the chromosomal rearrangement is a translocation, an inversion, or a duplication.

The entire contents of International Publication Number WO 2016/148994 is incorporated by reference herein.

### BRIEF DESCRIPTION OF THE DRAWINGS

FIGS. 1A-1F show live imaging of non-repetitive and repetitive loci. FIG. 1A shows a schematic of MUC4-targeted loci for pools of gRNAs (gRNAs). FIG. 1B shows co-localization of non-repetitive loci with a pool of 10 gRNAs with E3 (repetitive) loci in U2OS cells. 100% of 10 cells from 4 separate transfections showed co-localization. FIG. 1C shows a schematic of MUC4-targeted loci for non-repetitive single locus #72. FIG. 1D shows co-localization of non-repetitive single locus #72 using 1 gRNA and E3 (repetitive) loci. 93% of 44 cells from 10 separate transfections showed co-localization. FIG. 1E shows a schematic of MUC4-targeted loci for non-repetitive single locus #33. FIG. 1F shows co-localization of non-repetitive single locus #33 using 1 gRNA and E3 (repetitive) loci. 92% of 87 cells from 11 separate transfections showed co-localization. Scale bars are 5 μm. Arrow heads point to gRNAs bound to MUC4 loci.

FIGS. 2A-2H show live imaging of non-repetitive single loci. FIG. 2A shows a schematic of targeted MUC4 loci pairs using 1 gRNA each. The grid shows co-localization of loci pairs (shaded) in U2OS cells. FIG. 2B shows labeling of locus #1 and #12. 75% of 20 cells from 4 separate transfections showed co-localization. FIG. 2C shows labeling of locus #22 and locus #33. 100% of 10 cells from 2 transfections showed co-localization. FIG. 2D shows labeling of locus #33 and locus #72. 85% of 13 cells from 3 transfections showed co-localization. FIG. 2E shows labeling of locus #46 and #52. 100% of 10 cells from 3 transfections showed co-localization. FIG. 2F shows labeling of locus #56 and #60. 79% of 14 cells from 3 transfections showed co-localization. FIG. 2G shows labeling of locus #65 and #72. 63% of 16 cells from 3 transfections showed co-localization. FIG. 2H shows labeling of locus #72 and #1. 43% of 21 cells from 2 transfections showed co-localization. Scale bars are 5 μm. Arrow heads point to gRNAs bound to MUC4 loci.

FIGS. 3A-3J show that increasing the distance between gRNAs increases the distance between spots. FIG. 3A shows a schematic of targeted non-repetitive loci at increasing distances from non-repetitive locus #33 on chromosome 3. FIG. 3B shows the 3D distance between signals of one non-repetitive locus and non-repetitive locus #33 in U2OS cells (each spot represents one distance). The average is the horizontal line. n=17-36 loci pairs for each kilobase (kb). 8-16 cells were imaged for each kb. FIG. 3C shows labeling of locus 8 kb from locus #33. The average distance was 0.14 μm (n=30 pairs). FIG. 3D shows labeling of locus 14 kb from locus #33. The average distance was 0.16 μm (n=25 pairs). FIG. 3E shows labeling of locus 19 kb from locus #33. The average distance was 0.29 μm (n=30 pairs). FIG. 3F shows labeling of locus 24 kb from locus #33. The average distance was 0.51 μm (n=19 pairs). FIG. 3G shows labeling of locus 28 kb from locus #33. The average distance was 1.19 μm (n=20 pairs). FIG. 3H shows labeling of locus 44 kb from locus #33. The average distance was 1.21 μm (n=28 pairs). FIG. 3I shows labeling of locus 58.5 kb from locus #33. The average distance was 1.66 μm (n=36 pairs). FIG. 3J shows labeling of loci 74 kb from locus #33. The average distance was 1.74 μm (n=17 pairs). Scale bars are 5 μm.

FIGS. 4A-4E show live cell imaging of five sequential loci. FIG. 4A shows a schematic of Casilio sequential 0-28 kb-44 kb-58.5 kb-74 kb probes for visualizing a 74 kb genomic region of chr3:195,735,394-195809539 with gRNA targeting three locations (0, 44 kb, 74 kb) with 15×PBSc recruiting Clover-PUFc and gRNA targeting two location (28 kb, 58.5 kb) with 15×PBS9R recruiting PUF9R-mRuby2. FIGS. 4B and 4D show representative time-lapse images of chr3:195,735,394-195,809,539 imaged with 0-28 kb-44 kb-58.5 kb 74 kb Casilio probes in HEK293T(B) and ARPE-19(D) cells. (Scale bars, 1 □m). FIGS. 4C and 4E show 3D models of marked fluorescent clusters (see B and D) at time 0 in HEK293T(C) and ARPE-19(E) cells and the microscopic views from the x-y, y-z, and z-x planes.

FIGS. 5A-5D show live imaging of non-repetitive sequence #72. The co-localization of MUC4 non-repetitive single locus #72 using 1 gRNA and E3 locus (repetitive) in U2OS cells is shown. 93% of 44 cells from 10 transfections showed co-localization. Scale bars are 5 μm. Arrow heads point to gRNAs bound to MUC4 loci.

FIGS. 6A-6C show time-lapse imaging of non-repetitive sequence #72. FIGS. 6A-6B show co-localization of MUC4 non-repetitive single locus #72 using 1 gRNA and E3 repeats in U2OS cells. Images were taken every 30 minutes for 15 hours. FIG. 6C shows co-localization of MUC4 non-repetitive single locus #72 using 1 gRNA and E3 repeats. Images were taken every 30 minutes for 8 hours. Scale bars are 5 μm. Arrow heads point to gRNAs bound to MUC4 loci.

FIGS. 7A-7D show live imaging of non-repetitive sequence #33. FIGS. 7A-7D show co-localization of MUC4 non-repetitive single locus #33 using 1 gRNA and E3 repeats in U2OS cells. 92% of 87 cells from 11 transfections showed co-localization. Scale bars are 5 μm. Arrow heads point to gRNAs bound to MUC4 loci.

FIGS. 8A-8C show live imaging of non-repetitive CISTR-ACT single loci. FIG. 8A shows a schematic of CISTR-ACT targeted loci. FIGS. 8B-8C show co-localization of CISTR-ACT non-repetitive single locus #4 and #1 using 1 gRNA in U2OS cells. 100% of 12 cells from 4 transfections showed co-localization. Scale bars are 5 μm. Arrow heads point to gRNAs bound to CISTR-ACT loci.

FIGS. 9A-9G show live cell imaging of chromatin interactions. FIG. 9A shows a schematic of Casilio probes for visualizing chromatin interactions mediated by cohensin (RAD21) with a gRNA targeting locus A (genomic 5′ anchor) with 15×PBSc recruiting Clover-PUFc and a gRNA targeting locus B (genomic 3′ anchor) with 15×PBS9R recruiting PUF9R-mRuby2. FIG. 9B shows a UWash Genome Browser view of chr3:187318256-187680546 loop with anchors 367 kb apart on chromosome 3. Locus A and locus B indicate the gRNA binding locations. FIG. 9C shows representative time-lapse images of chr3:187318256-187680546 loop anchors, Locus A near MASP1 (green) and Locus B near BCL6 (magenta), in ARPE-19 cells. Image on the left shows the whole nucleus at time 0 (Scale bar, 5 μm). Image strips on the right show images of Pair 1 (upper) and Pair 2 (lower) at the indicated time point (Scale bars, 1 μm).

FIG. 9D shows a pairwise distance of fluorescent foci for Pair 1 and Pair 2 over time. FIG. 9E shows a UWash Genome Browser view of chr17:40302616-40355921 loop with anchors 55 kb apart on chromosome 17. FIG. 9F shows representative time-lapse images of chr17:40302616-40355921 loop anchors, Locus A near CDC6 (green) and Locus B near RARA (magenta), in ARPE-19 cells. Image on the left shows the whole nucleus at time 0 (Scale bar, 5 μm). Image strips on the right show images of Pair 1 (upper) and Pair 2 (lower) at the indicated time point (Scale bars, 1 μm). FIG. 9G shows a pairwise distance of fluorescent foci for Pair 1 and Pair 2 over time.

### DETAILED DESCRIPTION

Provided herein, in some aspects, are methods and compositions for imaging, in live cells, non-repetitive genomic loci using a catalytically-inactive RNA-guided nuclease (e.g., dCas9) and a single unique guide RNA (gRNA) per locus. These methods may be used, for example, to examine the local and global three-dimensional (3D) structure of different genes in real-time. As shown herein, the local 3D structure of a gene was examined using pairs of dual-labeling gRNAs that include an anchor gRNA and gRNAs designed to bind at increasing genomic distances relative to the anchor gRNA. The heterogeneity and dynamic nature of chromatin folding at the labeled locus was observed using this technique. The methods of the present disclosure address many of the technical challenges associated with the use of live cell imaging for studying nuclear processes, such as chromatin remodeling, especially in cells that are difficult to transfect. The methods also simplify genome-wide gRNA library design, as each target locus can be targeted with one gRNA, as compared to other approaches that require multiple gRNAs per target locus. Thus, the methods and compositions described herein, in some embodiments, facilitate perturbation of the (epi)genome (e.g., using activator and repressor modules) and concomitant read-out of 3D chromatin interaction dynamics (using the imaging modules described herein), offering a customizable and flexible technique to study, inter alia, nuclear architecture and processes.

### Live Cell Imaging

Chromatin conformation, localization, and dynamics are important for regulating cellular behaviors. While fluorescence in situ hybridization-based techniques have been widely used to investigate chromatin architectures in healthy and diseased conditions, the requirement for cell fixation has prohibited a comprehensive dynamic analysis of chromatin activities. More recently, dCas9-gRNA systems have been used to target non-repetitive loci, but these systems have been difficult to use for biological applications due to challenges in delivering dozens of gRNAs into cells and the accompanying increase in off-target effects associated with delivering such a large number of gRNAs (Chen B et al. Cell 2013; 155: 1479-1491; and Anton T. et al. Nucleus 2014; 5: 163-172).

The platform provided herein addresses these challenges by enabling multicolor labeling of non-repetitive (and/or low-repeat-containing) regions using a single unique gRNA per locus. The methods here use (a) a catalytically-inactive RNA-guided nuclease (e.g., dCas9), a unique RNA (gRNA) that comprises (i) a DNA-targeting sequence that is complementary to a non-repetitive genomic locus, (ii) a RNA-guided nuclease-binding sequence, and (iii) a Pumilio-FBF (PUF) domain-binding sequence, and (b) a detectable molecule (e.g., fluorescent protein) linked to a PUF domain (detectable conjugate) that binds to the PUF domain-binding sequence of the gRNA. In a live cell, the complex formed by interaction of the RNA-guided nuclease and the gRNA is guided to a specific non-repetitive genomic locus, where the gRNA serves as a docking site for the detectable conjugate. The detectable signal enables live-cell imaging at one or more non-repetitive genomic loci.

It should be understood that “unique gRNA” refers to a gRNA that binds to only one genomic locus (e.g., one chromatin locus) within a defined region, e.g., within a 1 kb region. That is, the unique gRNA is designed to include a DNA-targeting sequence that is complementary to only one other sequence within the defined region. In some embodiments, a unique gRNA is designed to bind to only one sequence in the entire genome of a cell. Nonetheless, as is known in the art, even though a gRNA is designed to be unique to a particular locus, it may bind “off-target,” in some instances.

In some aspects, the methods herein comprise imaging a live cell that comprises multiple genomic loci, each bound by a tripartite complex comprising a single unique gRNA bound by a detectable conjugate and a catalytically-inactive RNA-guided nuclease.

The live cell imaging (visualization) methods of the present disclosure, in some embodiments are used to image chromatin dynamics, for example, to examine organization of and changes to the genome. For example, the methods herein can be used to monitor multi-dimensional changes in chromatic structure by labeling multiple loci at increasing distances relative to an initial “anchor” gRNA and/or relative to each other.

In some embodiments, the methods are used to investigate the role of chromatin in transcriptional regulation. For example, the methods herein may be used to track chromatin loci (e.g., non-repetitive loci) throughout the cell cycle to determine differential positioning of transcriptionally active and inactive regions in the nucleus. In some embodiments, the methods may be used to image epigenetic regulation.

In some embodiments, the methods may be used to image (e.g., investigate, examine, etc.) processes associated with DNA replication, DNA damage repair, and/or gene expression.

In some embodiments, the methods are used to detect a chromosomal rearrangement in a cell. The methods may comprise, for example, delivering to a live cell (a) a catalytically-inactive RNA-guided nuclease, (b) a first single unique gRNA that comprises a DNA-targeting sequence that is designed to bind adjacent to and upstream from (5′ to) a nuclease cleavage site, (c) a detectable molecule linked to a PUF domain that binds to the PUF domain-binding sequence of the first gRNA, (d) a second single unique gRNA that comprises a DNA-targeting sequence that is designed to bind adjacent to and downstream from (3′ to) a nuclease cleavage site, and (e) a detectable molecule linked to a PUF domain that binds to the PUF domain-binding sequence of the second gRNA, wherein each gRNA further comprises a RNA-guided nuclease-binding sequence and a PUF domain-binding sequence. The methods may further comprise imaging in the live cell the distance between the first gRNA and the second gRNA to determine the presence or absence of a chromosomal rearrangement. A distance between the two gRNAs that is great than expected, for example, may indicate the presence of a chromosomal rearrangement. Alternatively, an expected distance between the two gRNAs may indicate the absence of a chromosomal rearrangement.

Various types of chromosomal rearrangements are known. In some embodiments, the chromosomal rearrangement is a translocation, an inversion, a duplication, or deletion.

Other aspects of the present disclosure provide methods for identifying a genetic abnormality in a cell, comprising delivering to a live cell (a) a catalytically-inactive RNA-guided nuclease, (b) a first single unique gRNA that comprises a DNA-targeting sequence that is designed to bind adjacent to and upstream from a genetic abnormality, (c) a detectable molecule linked to a PUF domain that binds to the PUF domain-binding sequence of the first gRNA, (d) a second single unique gRNA that comprises a DNA-targeting sequence that is designed to bind adjacent to and downstream from a genetic abnormality, and (e) a detectable molecule linked to a PUF domain that binds to the PUF domain-binding sequence of the second gRNA, wherein each gRNA further comprises a RNA-guided nuclease-binding sequence and a PUF domain-binding sequence. The methods may further comprise imaging in the live cell the distance between the first gRNA and the second gRNA to determine the presence or absence of a chromosomal rearrangement. In some embodiments, the genetic abnormality is a chromosomal rearrangement. In some embodiments, the chromosomal rearrangement is a translocation, an inversion, a duplication, or deletion.

In some aspects, the methods are used to detect multiple non-repetitive genomic loci (e.g., regions of chromatin) in live cells. For example, the methods may be used to detect 2-100, 2-75, 2-50, 2-25, 2-15, 2-10, 5-100, 5-75, 5-50, 5-25, 5-15, 5-10, 10-100, 10-75, 10-50, 10-25, or 10-15 non-repetitive loci. In some embodiments, the methods may be used to detect 2, 3, 4, 5, 6, 7, 8, 9, 10, 11, 12, 13, 14, 15, or more non-repetitive loci. Thus, in some embodiments, the live cells are transfected with 2-100, 2-75, 2-50, 2-25, 2-15, 2-10, 5-100, 5-75, 5-50, 5-25, 5-15, 5-10, 10-100, 10-75, 10-50, 10-25, or 10-15 unique gRNAs (or nucleic acids encoding the gRNAs). For example, live cells herein may be transfected with 2, 3, 4, 5, 6, 7, 8, 9, 10, 11, 12, 13, 14, 15, or more unique gRNAs (or nucleic acids encoding the gRNAs).

A single locus may be separated from any other locus by a distance of at least 1 kilobase pair (kb). In some embodiments, a single locus is separate from another locus by a distance of 1 kb to 100 kb. For example, a single locus may be separated from any other locus by a distance of 1-5 kb, 1-10 kb, 1-15 kb, 1-20 kb, 1-25 kb, 1-30 kb, 1-35 kb, 1-40 kb, 1-45 kb, 1-50 kb, 1-55 kb, 1-60 kb, 1-65 kb, 1-70 kb, 1-75 kb, 1-80 kb, 1-85 kb, 1-90 kb, 1-100 kb, 5-10 kb, 5-15 kb, 5-20 kb, 5-25 kb, 5-30 kb, 5-35 kb, 5-40 kb, 5-45 kb, 5-50 kb, 5-55 kb, 5-60 kb, 5-65 kb, 5-70 kb, 5-75 kb, 5-80 kb, 5-85 kb, 5-90 kb, 5-95 kb, 5-100 kb, 10-20 kb, 10-30 kb, 10-40 kb, 10-50 kb, 10-60 kb, 10-70 kb, 10-80 kb, 10-90 kb, 10-100 kb, 20-30 kb, 20-40 kb, 20-50 kb, 20-60 kb, 20-70 kb, 20-80 kb, 20-90 kb, 20-100 kb, 30-40 kb, 30-50 kb, 30-60 kb, 30-70 kb, 30-80 kb, 30-90 kb, 30-100 kb, 40-50 kb, 40-60 kb, 40-80 kb, 40-100 kb, 50-60 kb, 50-80 kb, 50-100 kb, 60-70 kb, 60-80 kb, 60-100 kb, 70-80 kb, 70-90 kb, 70-100 kb, 80-90 kb, 80-100 kb, or 90-100 kb. In some embodiments, the distance between at least two of the non-repetitive genomic loci is 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, or more. In some embodiments, the gRNAs are not pooled, i.e., the gRNAs are not directed to the same genomic locus.

In some embodiments, the loci labeled are located at increasing distances relative to an “anchor” locus. An anchor locus is simply a known fixed locus that is labeled as provided herein. Other labeled loci may be characterized as being located a certain distance from an anchor locus. As shown in the Example, for example, gRNAs herein may be designed to bind at increasing genomic distances relative to the anchor gRNA. In this way, multiple loci within a certain genomic region can be labeled, imaged, and characterized relative to one other, to provide information, for example, about dynamic chromatin interactions in that genomic region. For example, a first locus may be located at a distance of 1 kb from an anchor locus, a second locus may be located at a distance of 2 kb from the anchor locus (e.g., 1 kb from the first locus), a third locus may be located at a distance of 3 kb from the anchor locus (e.g., 1 kb from the second locus, and 2 kb from the first locus), and so on.

A detectable molecule may be, for example, a fluorescent protein, a fluorophore, or other fluorescent molecule. The detectable molecules used herein may be the same or different, relative to one another. For example, all detectable molecules in a single cell may be a green fluorescent protein (GFP), each localized to a single locus, or multiple different fluorescent proteins may be used (e.g., red, green, blue, yellow; each color localized to a single locus). Thus, in some embodiments, fluorescent proteins having different emission wavelengths relative to one another may be used. In some embodiments, 2, 3, 4, 5, 6, 7, 8, 9, or 10 different detectable molecules (e.g., different fluorescent proteins) may be used. Non-limiting examples of fluorescent proteins that may be used herein include GFP, Clover, mRuby2, Superfolder GFP, EGFP, BFP, EBFP, EBFP2, Azurite, mKalama1, CFP, ECFP, Cerulean, CyPet, mTurquoise2, YFP, Citrine, Venus, Ypet, BFPms1, roGFP, and bilirubin-inducible fluorescent proteins such as UnaG, dsRed, eqFP611, Dronpa, TagRFPs, KFP, EosFP, Dendra, IrisFP. Other fluorescent proteins may be used.

Imaging may occur 12-96 hours post-transfection. For example, imaging may occur 12, 24, 36, 48, 60, 72, 84, or 96 hours after transfection. As another example, imaging may occur 12-24, 12-48, 12-72, 24-48, 24-72, or 48-72 hours post-transfection. Imaging may occur for less than 1, 2, 3, 4, 5, 6, 7, 8, 9, 10, 11, 12, 13, 14, or 15 minutes. In some embodiments, images are taken at certain time points, for example, every 1, 2, 3, 4, 5, 6, 7, 8, 9, 10, 15, 20, 25, 30, 35, 40, 45, 50, 55, or 60 seconds. In some embodiments, images are taken every 1, 2, 3, 4, 5, 6, 7, 8, 9, 10, 15, 20, 25, 30, 35, 40, 45, 50, 55, or 60 minutes. In some embodiments, imaging takes place over a period of 1, 2, 3, 4, 5, 6, 7, 8, 9, 10, 11, 12, 14, 16, 18, 20, 24, 36, 48, 60, or 72 hours. For example, images may be captured every 30 minutes for 5, 6, 7, 8, 9, 10, 11, 12, 13, 14, 15, 16, 17, 18, 19, or 20 hours.

Imaging may be accomplished by any method known in the art. The method of imaging selected depends on the detectable molecule used. For example, fluorescent microscopy (e.g., confocal fluorescent microscopy) can be used to examine the live cell populations when a fluorescent detectable molecule is used.

### RNA-Guided Nuclease

Methods described herein include the use of an RNA-guided nuclease, such as a catalytically-inactive RNA-guided nuclease. The catalytically-inactive RNA-guided nuclease is engineered to have reduced or deficient nuclease activity, but retains its DNA-binding ability when complexed with the gRNA. Examples of RNA-guided nucleases include Cpf1, Cas9, and active fragments, derivatives, and variants thereof. In one embodiment, the catalytically-inactive RNA-guided nuclease is a modified Cas9 protein, such as dead Cas9 (dCas9) protein. In some embodiments, the dCas9 has substantially no detectable endonuclease (e.g., endodeoxyribonuclease) activity. In some embodiments when a dCas9 has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the polypeptide can still bind to target DNA in a site-specific manner, because it is still guided to a target polynucleotide sequence by a DNA-targeting sequence of gRNA, as long as it retains the ability to interact with the Cas9-binding sequence of the gRNA.

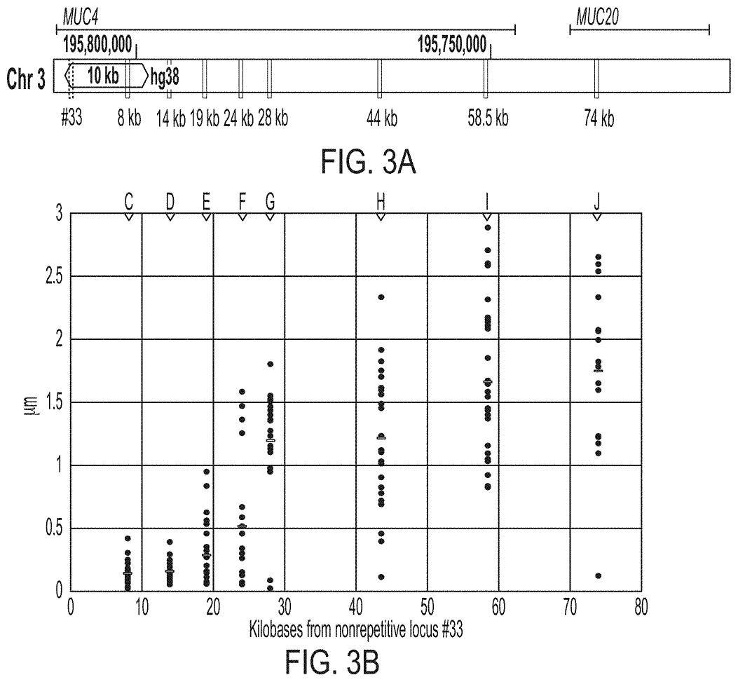

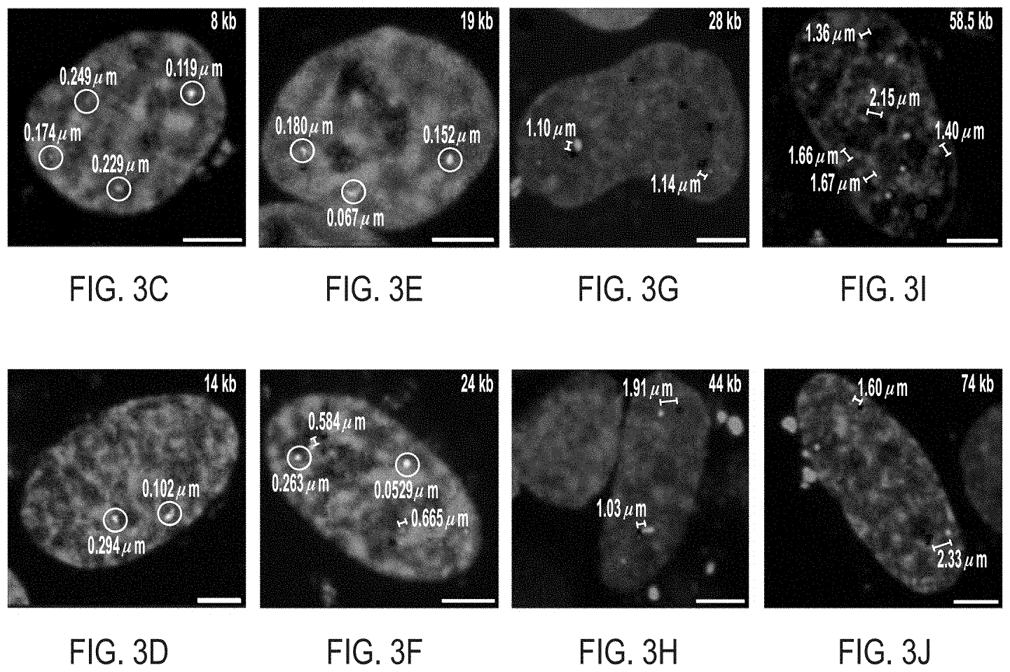

In some cases, the dCas9 has a reduced ability to cleave both the complementary and the non-complementary strands of the target DNA. As a non-limiting example, in some cases, the dCas9 harbors both D10A and H840A mutations of the amino acid sequence depicted in FIG. 3 of WO 2013/176772 or the corresponding mutations of any of the amino acid sequences set forth in SEQ ID NOs: 1-256 and 795-1346 of WO 2013/176772 (all such sequences incorporated by reference).

Guide RNA (gRNA)

The RNA-guided nuclease interacts with an engineered guide RNA (gRNA), such as a unique single gRNA. The unique single gRNA described herein comprises at least three components: a DNA-targeting sequence, an RNA-guided nuclease-binding sequence, and an RNA-binding protein (RBP) domain-binding sequence. In some embodiments, the three segments are arranged in that order, from 5′ to 3′.

The RNA-guided nuclease-binding sequence of the gRNA and the catalytically-inactive ribonucleic acid (RNA)-guided nuclease (e.g., dCas9 protein) can form a complex that binds to a specific target polynucleotide sequence, based on the sequence complementarity between the DNA-targeting sequence and the target polynucleotide sequence. The DNA-targeting sequence of the gRNA provides target specificity to the complex via its sequence complementarity to the target polynucleotide sequence of a target DNA, as discussed below.

DNA-Targeting Sequence

The DNA-targeting sequence comprises a nucleotide sequence that is complementary to a specific sequence within a target DNA (or the complementary strand of the target DNA). In other words, the DNA-targeting sequence interacts with a target polynucleotide sequence of the target DNA in a sequence-specific manner via hybridization (i.e., base pairing). As such, the nucleotide sequence of the DNA-targeting sequence may vary, and it determines the location within the target DNA that the gRNA and the target DNA will interact. The DNA-targeting sequence can be modified or designed (e.g., by genetic engineering) to hybridize to any desired sequence within the target DNA. In some embodiments, the DNA-targeting sequence is complementary to a sequence within a non-repetitive genomic locus, for example, the DNA-targeting sequence targets a chromatin sequence. In some embodiments, the target polynucleotide sequence is immediately 3′ to a PAM (protospacer adjacent motif) sequence of the complementary strand, which can be 5′-CCN-3′, wherein N is any DNA nucleotide. That is, in this embodiment, the complementary strand of the target polynucleotide sequence is immediately 5′ to a PAM sequence that is 5′-NGG-3′, wherein N is any DNA nucleotide. In related embodiments, the PAM sequence of the complementary strand matches the catalytically-inactive RNA-guided nuclease (e.g., dCas9).

The DNA-targeting sequence can have a length of from about 12 nucleotides to about 100 nucleotides. For example, the DNA-targeting sequence can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the DNA-targeting sequence can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

The nucleotide sequence of the DNA-targeting sequence that is complementary to a target polynucleotide sequence of the target DNA can have a length of at least about 12 nt. For example, the DNA-targeting sequence that is complementary to a target polynucleotide sequence of the target DNA can have a length at least about 12 nt, at least about 15 nt, at least about 18 nt, at least about 19 nt, at least about 20 nt, at least about 25 nt, at least about 30 nt, at least about 35 nt or at least about 40 nt. For example, the DNA-targeting sequence that is complementary to a target polynucleotide sequence of a target DNA can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. The nucleotide sequence of the DNA-targeting sequence that is complementary to the target polynucleotide sequence of the target DNA can have a length of at least about 12 nt.

In some cases, the DNA-targeting sequence that is complementary to a target polynucleotide sequence of the target DNA is 20 nucleotides in length. In some cases, the DNA-targeting sequence that is complementary to a target polynucleotide sequence of the target DNA is 19 nucleotides in length.

The percent complementarity between the DNA-targeting sequence and the target polynucleotide sequence of the target DNA can be at least 50% (e.g., at least 55%, at least 60%, at least 65%, at least 70%, at least 75%, at least 80%, at least 85%, at least 90%, at least 95%, at least 97%, at least 98%, at least 99%, or 100%). In some cases, the percent complementarity between the DNA-targeting sequence and the target polynucleotide sequence is 100% over the seven or eight contiguous 5′-most nucleotides of the target polynucleotide sequence. In some cases, the percent complementarity between the DNA-targeting sequence and the target polynucleotide sequence is at least 60% over about 20 contiguous nucleotides. In some cases, the percent complementarity between the DNA-targeting sequence and the target polynucleotide sequence is 100% over the 7, 8, 9, 10, 11, 12, 13, or 14 contiguous 5′-most nucleotides of the target polynucleotide sequence (i.e., the 7, 8, 9, 10, 11, 12, 13, or 14 contiguous 3′-most nucleotides of the DNA-targeting sequence), and as low as 0% over the remainder. In such a case, the DNA-targeting sequence can be considered to be 7, 8, 9, 10, 11, 12, 13, or 14 nucleotides in length, respectively.

RNA-Guided Nuclease-Binding Sequence

The RNA-guided nuclease-binding sequence of the gRNA binds to the catalytically-inactive RNA-guided nuclease (e.g., dCas9). The catalytically-inactive RNA-guided nuclease and RNA-guided nuclease-binding sequence of the gRNA together bind to the target polynucleotide sequence recognized by the DNA-targeting sequence. The RNA-guided nuclease-binding sequence comprises two complementary stretches of nucleotides that hybridize to one another to form a double stranded RNA duplex (a dsRNA duplex). These two complementary stretches of nucleotides may be covalently linked by intervening nucleotides known as linkers or linker nucleotides (e.g., in the case of a single-molecule polynucleotide), and hybridize to form the double stranded RNA duplex (dsRNA duplex, or “Cas9-binding hairpin”) of the Cas9-binding sequence, thus resulting in a stem-loop structure.

The RNA-guided nuclease-binding sequence can have a length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the RNA-guided nuclease-binding sequence can have a length of from about 15 nucleotides (nt) to about 80 nt, from about 15 nt to about 50 nt, from about 15 nt to about 40 nt, from about 15 nt to about 30 nt, from about 37 nt to about 47 nt (e.g., 42 nt), or from about 15 nt to about 25 nt.

The dsRNA duplex of the RNA-guided nuclease-binding sequence can have a length from about 6 base pairs (bp) to about 50 bp. For example, the dsRNA duplex of the Cas9-binding sequence can have a length from about 6 bp to about 40 bp, from about 6 bp to about 30 bp, from about 6 bp to about 25 bp, from about 6 bp to about 20 bp, from about 6 bp to about 15 bp, from about 8 bp to about 40 bp, from about 8 bp to about 30 bp, from about 8 bp to about 25 bp, from about 8 bp to about 20 bp or from about 8 bp to about 15 bp. For example, the dsRNA duplex of the RNA-guided nuclease-binding sequence can have a length from about from about 8 bp to about 10 bp, from about 10 bp to about 15 bp, from about 15 bp to about 18 bp, from about 18 bp to about 20 bp, from about 20 bp to about 25 bp, from about 25 bp to about 30 bp, from about 30 bp to about 35 bp, from about 35 bp to about 40 bp, or from about 40 bp to about 50 bp. In some embodiments, the dsRNA duplex of the RNA-guided nuclease-binding sequence has a length of 36 base pairs. The percent complementarity between the nucleotide sequences that hybridize to form the dsRNA duplex of the RNA-guided nuclease-binding sequence can be at least about 60%. For example, the percent complementarity between the nucleotide sequences that hybridize to form the dsRNA duplex of the RNA-guided nuclease-binding sequence can be at least about 65%, at least about 70%, at least about 75%, at least about 80%, at least about 85%, at least about 90%, at least about 95%, at least about 98%, or at least about 99%. In some cases, the percent complementarity between the nucleotide sequences that hybridize to form the dsRNA duplex of the RNA-guided nuclease-binding sequence is 100%.

The linker can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker is 4 nt.

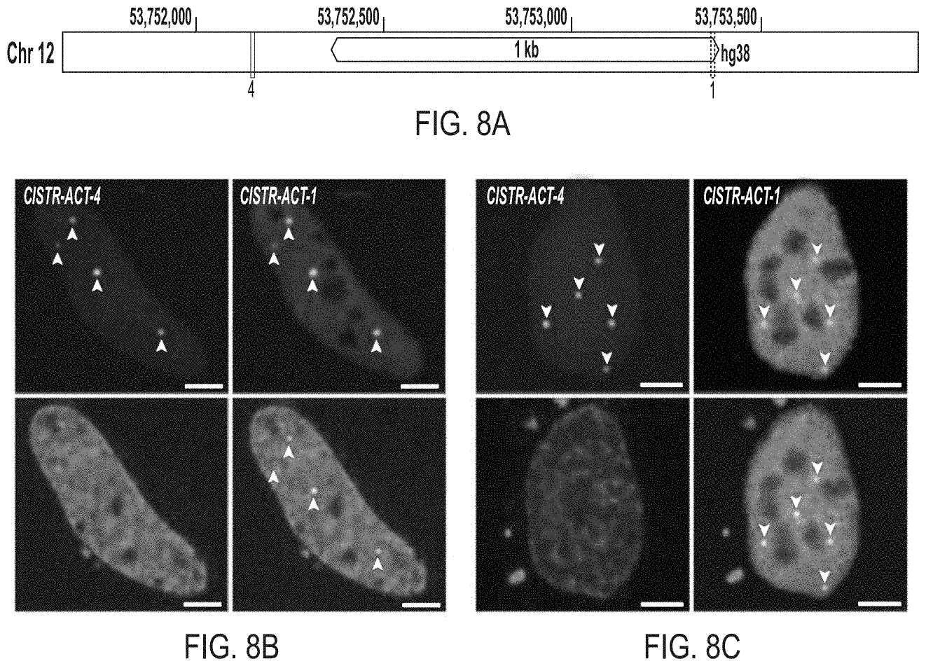

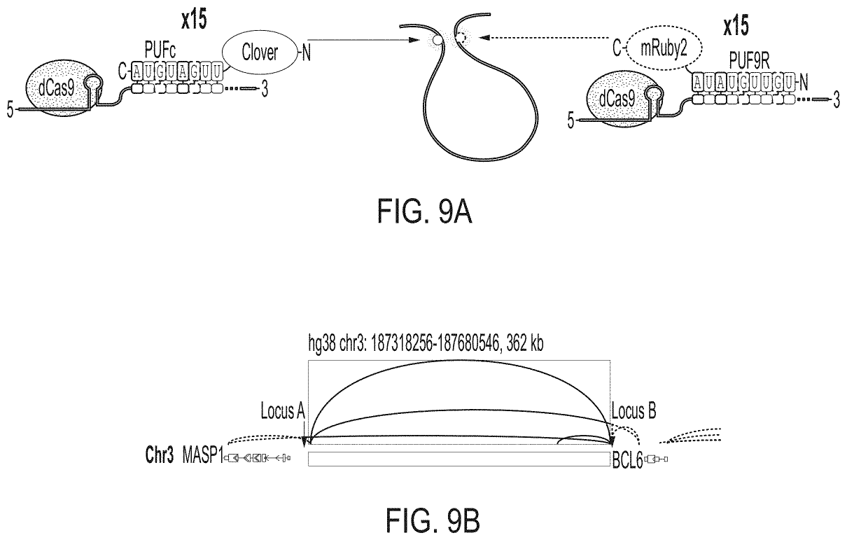

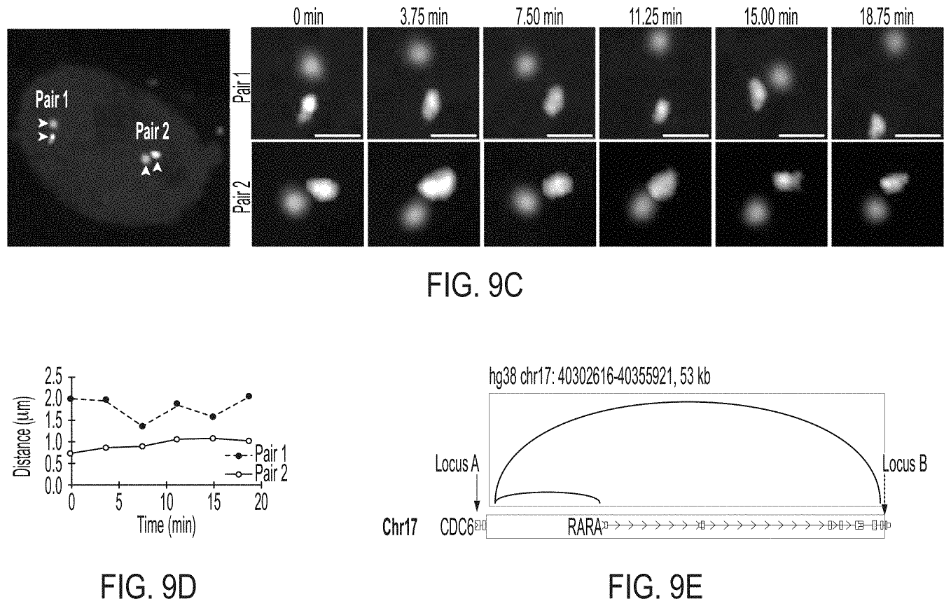

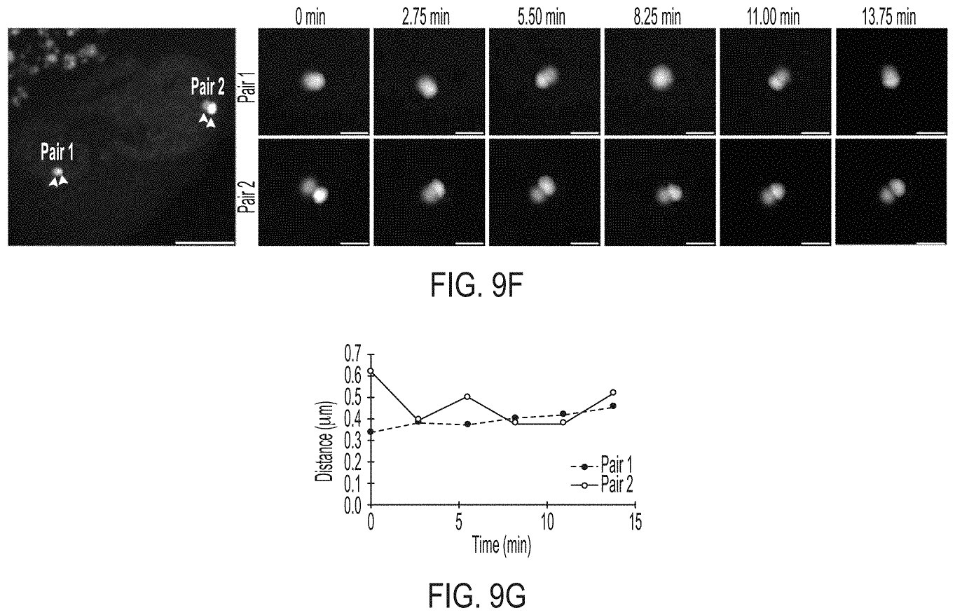

Non-limiting examples of nucleotide sequences that can be included in a suitable RNA-guided nuclease-binding sequence (i.e., Cas9 handle) are set forth in SEQ ID NOs: 563-682 of WO 2013/176772 (see, for examples, FIGS. 8 and 9 of WO 2013/176772), incorporated herein by reference.

In some cases, a suitable RNA-guided nuclease-binding sequence comprises a nucleotide sequence that differs by 1, 2, 3, 4, or 5 nucleotides from any one of the above-listed sequences.

### RNA-Binding Protein (RBP) Domain Binding Sequence(s)

The gRNA comprises one or more tandem sequences, each of which can be specifically recognized and bound by a specific RNA-binding protein domain (e.g., a Pumilio-FBF (PUF) domain). Such sequences, referred to herein as RNA-binding protein (RBP) domain-binding sequences (e.g., PUF domain-binding sequences, PBS), may be engineered to bind any RBP binding domain (e.g., PUF domain). For example, based on the nucleotide-specific interaction between the individual PUF motifs of PUF domain and the single RNA nucleotide they recognize, the PBS sequences can be any designed sequences that bind their corresponding PUF domain.

In some embodiments, a PBS of the present disclosure has 8-mer. In other embodiments, a PBS of the present disclosure has 5, 6, 7, 8, 9, 10, 11, 12, 13, 14, 15, 16 or more RNA nucleotides.

In some embodiments, the PBS of the present disclosure has the sequence 5′-UGUAUAUA-3′, and binds the wild-type human Pumilio 1 PUF domain. In some embodiments, the PBS of the present disclosure has the sequence 5′-UGUAUGUA-3′, and binds the PUF domain PUF(3-2). In some embodiments, the PBS of the present disclosure has the sequence 5′-UUGAUAUA-3′, and binds the PUF domain C. In some embodiments, the PBS of the present disclosure has the sequence 5′-UGGAUAUA-3′, and binds the PUF domain PUF(6-2). In some embodiments, the PBS of the present disclosure has the sequence 5′-UUUAUAUA-3′, and binds the PUF domain PUF(7-2). In some embodiments, the PBS of the present disclosure has the sequence 5′-UGUGUGUG-3′, and binds the PUF domain PUF⁵³¹. In some embodiments, the PBS of the present disclosure has the sequence 5′-UGUAUAUG-3′, and binds the PUF domain PUF(1-1). In some embodiments, the PBS of the present disclosure has the sequence 5′-UUUAUAUA-3′ or 5′-UAUAUAUA-3′, and binds the PUF domain PUF(7-1). In some embodiments, the PBS of the present disclosure has the sequence 5′-UGUAUUUA-3′, and binds the PUF domain PUF(3-1). In some embodiments, the PBS of the present disclosure has the sequence 5′-UUUAUUUA-3′, and binds the PUF domain PUF(7-2/3-1). In some embodiments, the PBS of the present disclosure has the sequence 5′-UUGAUGUA-3′ and binds the PUF domain PUFc. In some embodiments, the PBS of the present disclosure has the sequence 5′-UGUUGUAUA-3′ and binds the PUF domain PUF9R. Any one of the PUF domains described in WO 2016/148994 may be used as provided herein. Other PUF domains may be used.

In some embodiments, one or more spacer region(s) separates two adjacent PBS sequences. The spacer regions may have a length of from about 3 nucleotides to about 100 nucleotides. For example, the spacer can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the spacer can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the spacer is 4 nt.

### Detectable Conjugates

In order to image the targeted non-repetitive locus or loci, at least one detectable molecule is required. In some embodiments, an RNA-binding protein (RBP) domain sequence (e.g., a PUF domain sequence) is linked to a detectable molecule (referred to herein as a detectable conjugate), which may be used for imaging live cells. The detectable molecules, in some embodiments, may be fluorescent proteins, polypeptides, variants, or functional domains thereof, such as GFP, Clover, mRuby2, Superfolder GFP, EGFP, BFP, EBFP, EBFP2, Azurite, mKalama1, CFP, ECFP, Cerulean, CyPet, mTurquoise2, YFP, Citrine, Venus, Ypet, BFPms1, roGFP, and bilirubin-inducible fluorescent proteins such as UnaG, dsRed, eqFP611, Dronpa, TagRFPs, KFP, EosFP, Dendra, IrisFP, etc. In some embodiments, the detectable molecules are fluorophores. Other detectable molecules may be used.

The RBP domain, linked to the detectable molecule, hybridizes with the RBP domain binding sequence of the gRNA. The detectable molecule can then be imaged, indicating the target non-repetitive locus or loci. The RBP domain sequence, in some embodiments, is a PUF domain.

PUF proteins (named after Drosophila Pumilio and C. elegans fern-3 binding factor) are known to be involved in mediating mRNA stability and translation. These protein contain a unique RNA-binding domain known as the PUF domain. The RNA-binding PUF domain, such as that of the human Pumilio 1 protein (referred here also as PUM), contains 8 repeats (each repeat called a PUF motif or a PUF repeat) that bind consecutive bases in an anti-parallel fashion, with each repeat recognizing a single base—i.e., PUF repeats R1 to R8 recognize nucleotides N8 to N1, respectively. For example, PUM is composed of eight tandem repeats, each repeat consisting of 34 amino acids that folds into tightly packed domains composed of alpha helices. In some embodiments, the RBP domain-detectable molecule construct comprises 1, 2, 3, 4, 5, 6, 7, 8, 9, 10 or more PUF domains.

Each PUF repeat uses two conserved amino acids from the center of each repeat to specifically recognize the edge of one individual base within the RNA recognition sequence, and a third amino acid (Tyr, His or Arg) to stack between adjacent bases, causing a very specific binding between a PUF domain and an 8-mer RNA. For example, the code to recognize base U is the amino acid sequence “NYxxQ”, whereas “(C/S)RxxQ” recognizes A and “SNxxE” recognizes G. These amino acids correspond to positions 12, 13, and 16 in the human Pumilio 1 PUF motif. The two recognition amino acid side chains at positions 12 and 16 in each PUF α-α-α repeat recognize the Watson-Crick edge of the corresponding base and largely determine the specificity of that repeat.

Therefore, the sequence specificity of the PUF domains can be altered precisely by changing the conserved amino acid (e.g., by site-directed mutagenesis) involved in base recognition within the RNA recognition sequence. By changing two amino acids in each repeat, a PUF domain can be modified to bind almost any 8-nt RNA sequence. This unique binding system makes PUF and its derivatives a programmable RNA-binding domain that can be engineered, in some embodiments, to bind a specific PUF domain binding sequence in the gRNA, and therefore, bringing the detection molecule to a specific PBS on the gRNA.

As used herein, “PUF domain” refers to a wildtype or naturally existing PUF domain, as well as a PUF homologue domain that is based on/derived from a natural or existing PUF domain, such as the prototype human Pumilio 1 PUF domain. The PUF domain of the present disclosure specifically binds to an RNA sequence (e.g., an 8-mer RNA sequence), wherein the overall binding specificity between the PUF domain and the RNA sequence is defined by sequence specific binding between each PUF motif/PUF repeat within the PUF domain and the corresponding single RNA nucleotide.

In some embodiments, the PUF domain comprises or consists essentially of 8 PUF motifs, each specifically recognizes and binds to one RNA nucleotide (e.g., A, U, G, or C).

In some embodiments, the PUF domain has more or less than 8 PUF motifs/repeats, e.g., the PUF domain comprises or consists essentially of 5, 6, 7, 8, 9, 10, 11, 12, 13, 14, 15, 16, or more PUF repeats/motifs, each specifically recognizes and binds to one RNA nucleotide (e.g., A, U, G, or C), so long as the PUF domain binds the RNA of 5, 6, 7, 8, 9, 10, 11, 12, 13, 14, 15, 16 or more nucleotides. By increasing or decreasing the number of PUF motifs, the length of the recognized RNA will be correspondingly increased or decreased. Since each PUF motif recognizes one RNA base, decreasing the domain by one motif decreases the length of the RNA recognized by one base; while increasing the domain by one motif increases the length of the RNA recognized by one base. Any number of motifs may be present. Therefore, in such embodiments, the specificity of the PUF domain-fusions of the present disclosure may be altered due to changes in PUF domain length. In some embodiments, the additional PUF motifs are inserted between two of the original PUF motifs, e.g., before the 1^{st}, between the 1^{st} and the 2^{nd}, the 2^{nd} and the 3^{rd}, the 3^{rd}, and the 4^{th}, the 4^{th} and the 5^{th}, the 5^{th} and the 6^{th}, the 6^{th} and the 7^{th}, the 7th and the 8^{th}, or after the 8^{th}. In some embodiments, there are 1, 2, 3, 4, 5, 6, 7, 8, or more inserted PUF motifs between any of the insertion points above. For example, in some embodiments, there are 1, 2, 3, 4, 5, 6, 7, 8, or more inserted PUF motifs between the 5^{th} and the 6^{th} original PUF motif. Filipovska et al. (Nature Chemical Biology doi: 10.1038/NChemBio.577, published online: 15 May 2011) have reported an engineered PUF domain with 16 PUF motifs, including 8 additional PUF motifs inserted between the 5^{th} and 6^{th} original PUF motifs.

In some embodiments, the PUF domain comprises PUF motifs from different PUF domains from different proteins. For example, a PUF domain of the present disclosure may be constructed with PUF motifs from the human Pumilio 1 protein and one or more other PUF motifs from one or more other PUF proteins, such as PuDp or FBF. The RNA binding pockets of PUF domains have natural concave curvatures. Since different PUF proteins may have different curvatures, different PUF motifs in a PUF domain may be used to alter the curvature of the PUF domain. Altering the curvature is another method for altering the specificity and/or binding affinity of the PUF domain since flatter curvatures may allow for the recognition of more RNA bases.

Also included in the scope of the present disclosure are functional variants of the subject PUF domains or fusions thereof. The term “functional variant” as used herein refers to a PUF domain having substantial or significant sequence identity or similarity to a parent PUF domain, which functional variant retains the biological activity of the PUF domain of which it is a variant—e.g., one that retains the ability to recognize target RNA to a similar extent, the same extent, or to a higher extent in terms of binding affinity, and/or with substantially the same or identical binding specificity, as the parent PUF domain. The functional variant PUF domain can, for instance, be at least about 30%, 50%, 75%, 80%, 90%, 98% or more identical in amino acid sequence to the parent PUF domain. The functional variant can, for example, comprise the amino acid sequence of the parent PUF domain with at least one conservative amino acid substitution, for example, conservative amino acid substitutions in the scaffold of the PUF domain (i.e., amino acids that do not interact with the RNA). Alternatively or additionally, the functional variants can comprise the amino acid sequence of the parent PUF domain with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent PUF domain, or may alter the stability of the PUF domain to a desired level (e.g., due to substitution of amino acids in the scaffold). The PUF domain can consist essentially of the specified amino acid sequence or sequences described herein, such that other components, e.g., other amino acids, do not materially change the biological activity of the functional variant.

In some embodiments, the PUF domain is a Pumilio homology domain (PU-HUD). In a particular embodiment, the PU-HUD is a human Pumilio 1 domain. The sequence of the human PUM is known in the art and is reproduced below (SEQ ID NO: 53):

Gly Arg Ser Arg Leu Leu Glu Asp Phe Arg Asn Asn
Arg Tyr Pro Asn Leu Gln Leu Arg Glu Ile Ala Gly
His Ile Met Glu Phe Ser Gln Asp Gln His Gly Ser
Arg Phe Ile Gln Leu Lys Leu Glu Arg Ala Thr Pro
Ala Glu Arg Gln Leu Val Phe Asn Glu Ile Leu Gln
Ala Ala Tyr Gln Leu Met Val Asp Val Phe Gly Asn
Tyr Val Ile Gln Lys Phe Phe Glu Phe Gly Ser Leu
Glu Gln Lys Leu Ala Leu Ala Glu Arg Ile Arg Gly
His Val Leu Ser Leu Ala Leu Gln Met Tyr Gly Cys
Arg Val Ile Gln Lys Ala Leu Glu Phe Ile Pro Ser
Asp Gln Gln Asn Glu Met Val Arg Glu Leu Asp Gly
His Val Leu Lys Cys Val Lys Asp Gln Asn Gly Asn
His Val Val Gln Lys Cys Ile Glu Cys Val Gln Pro
Gln Ser Leu Gln Phe Ile Ile Asp Ala Phe Lys Gly
Gln Val Phe Ala Leu Ser Thr His Pro Tyr Gly Cys
Arg Val Ile Gln Arg Ile Leu Glu His Cys Leu Pro
Asp Gln Thr Leu Pro Ile Leu Glu Glu Leu His Gln
His Thr Glu Gln Leu Val Gln Asp Gln Tyr Gly Asn
Tyr Val Ile Gln His Val Leu Glu His Gly Arg Pro
Glu Asp Lys Ser Lys Ile Val Ala Glu Ile Arg Gly
Asn Val Leu Val Leu Ser Gln His Lys Phe Ala Ser
Asn Val Val Glu Lys Cys Val Thr His Ala Ser Arg
Thr Glu Arg Ala Val Leu Ile Asp Glu Val Cys Thr
Met Asn Asp Gly Pro His Ser Ala Leu Tyr Thr Met
Met Lys Asp Gln Tyr Ala Asn Tyr Val Val Gln Lys
Met Ile Asp Val Ala Glu Pro Gly Gln Arg Lys Ile
Val Met His Lys Ile Arg Pro His Ile Ala Thr Leu
Arg Lys Tyr Thr Tyr Gly Lys His Ile Leu Ala Lys
Leu Glu Lys Tyr Tyr Met Lys Asn Gly Val Asp Leu
Gly

The wt human PUM specifically binds the Nanos Response Element (NRE) RNA, bearing a core 8-nt sequence 5′-UGUAUAUA-3′.

In some embodiments, the PUF domain of the present disclosure is any PUF protein family member with a Pum-HD domain. Non-limiting examples of a PUF family member include FBF in C. elegans, Ds pum in Drosophila, and PUF proteins in plants such as Arabidopsis and rice. A phylogenetic tree of the PUM-HDs of Arabidopsis, rice and other plant and non-plant species is provided in Tam et al. (“The Puf family of RNA-binding proteins in plants: phylogeny, structural modeling, activity and subcellular localization.” BMC Plant Biol. 10:44, 2010, the entire contents of which are incorporated by reference herein).

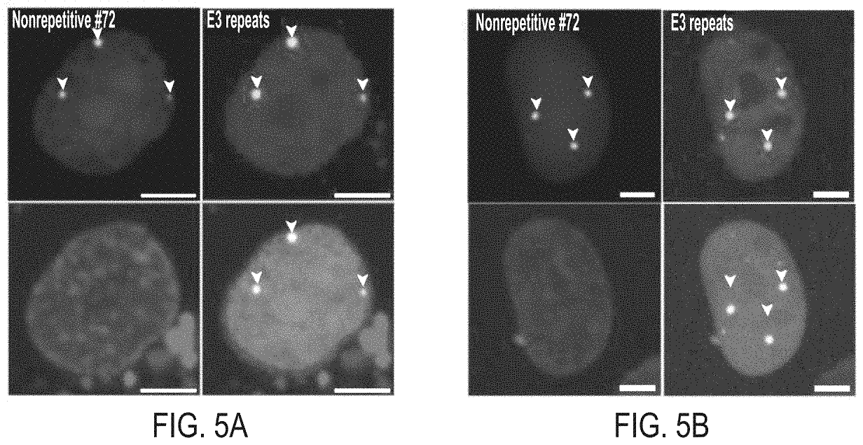

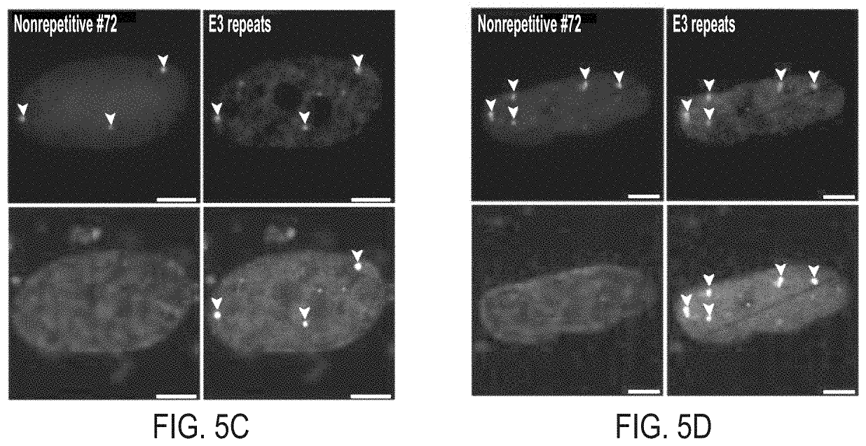

PUF family members are highly conserved from yeast to human, and all members of the family bind to RNA in a sequence specific manner with a predictable code. The accession number for the domain is PS50302 in the Prosite database (Swiss Institute of Bioinformatics) and a sequence alignment of some of the members of this family is shown in FIGS. 5 & 6 of WO 2011-160052 A2 (ClustalW multiple sequence alignment of human, mouse, rat Pumilio 1 (hpum1, Mpum1, Ratpum1) and human and mouse Pumilio 2 (hpum2, Mpum2), respectively.

Any of the subject PUF domain can be made using, for example, a Golden Gate Assembly kit (see Abil et al., Journal of Biological Engineering 8:7, 2014), which is available at Addgene (Kit #1000000051).

### Cells

As discussed above, the methods described herein may be used to image live cells (e.g., in vivo, in vitro, and/or in situ). Because the gRNA provides specificity by hybridizing to target polynucleotide sequence of a target DNA, the cells include, but are not limited to, a bacterial cell; an archaeal cell; a single-celled eukaryotic organism; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell; an animal cell; a cell from an invertebrate animal (e.g., an insect, a cnidarian, an echinoderm, a nematode, etc.); a eukaryotic parasite (e.g., a malarial parasite, e.g., Plasmodium falciparum; a helminth; etc.); a cell from a vertebrate animal (e.g., fish, amphibian, reptile, bird, mammal); a mammalian cell, e.g., a rodent cell, a human cell, a non-human primate cell, etc. Suitable cells for imaging include naturally-occurring cells; genetically modified cells (e.g., cells genetically modified in a laboratory, e.g., by the “hand of man”); and cells manipulated in vitro in any way. In some embodiments, a cell is isolated or cultured.

Any type of cell may be of interest (e.g., a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells,” “primary cell lines,” and “primary cultures” are used interchangeably herein to refer to cells and cells cultures that have been derived from a subject and allowed to grow in vitro for a limited number of passages, i.e. splittings, of the culture. For example, primary cultures include cultures that may have been passaged 0 times, 1 time, 2 times, 4 times, 5 times, 10 times, or 15 times, but not enough times go through the crisis stage. Primary cell lines can be maintained for fewer than 10 passages in vitro. In some embodiments, the cells are grown in culture.

If the cells are primary cells, such cells may be harvested from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g. normal saline, phosphate-buffered saline (PBS), Hank's balanced salt solution, etc., conveniently supplemented with fetal calf serum or other naturally occurring factors, in conjunction with an acceptable buffer at low concentration, e.g., from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% dimethyl sulfoxide (DMSO), 50% serum, 40% buffered medium, or other solutions commonly used in the art to preserve cells at such freezing temperatures, and thawed in a manner as commonly known in the art for thawing frozen cultured cells.

Introduction of gRNA, RNA-Guided Nuclease, and Detectable Molecule Construct into Cells

The gRNA, RNA-guided nuclease (e.g., dCas9), and detectable molecule construct (e.g., detectable molecule linked to an RBP domain) can be introduced into a cell by any of a variety of well-known methods.

Methods of introducing a nucleic acid into a cell are known in the art, and any known method can be used to introduce a nucleic acid (e.g., vector or expression construct) into a target cell. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv. Drug Deliv. Rev., pii: S0169-409X(12)00283-9.doi:10.1016/j.addr.2012.09.023), and the like. In one embodiment, the gRNA, RNA-guided nuclease (e.g., dCas9), and detectable molecule construct (e.g., detectable molecule linked to an RBP domain) are introduced into the cell via transfection.

Thus, the present disclosure also provides an isolated nucleic acid comprising a nucleotide sequence encoding the gRNA. In some cases, the isolated nucleic acid also comprises a nucleotide sequence encoding an RNA-guided nuclease (e.g., dCas9).

In one embodiment, the dCas9, the gRNA containing PUF binding sites, and PUF-detectable molecule construct are cloned into separate plasmids. The plasmids may then be linearized using any method known in the art (e.g., with BglII), and then subjected to in vitro transcription. The resulting RNA is then used to transfect the cells. In some embodiments, more than one gRNA is used (e.g., to detect multiple loci). In these instances, each gRNA may be added in equal amounts (e.g., 33 ng of each gRNA), or in unequal amounts (e.g., 33 ng of one gRNA, and 67 ng of a different gRNA).

In some embodiments, a subject method involves introducing into a cell (or a population of cells) one or more nucleic acids (e.g., vectors) comprising nucleotide sequences encoding a single unique gRNA and/or a RNA-guided nuclease (e.g., dCas9 protein) and/or a detectable molecule construct (e.g., a PUF domain linked to a fluorescent protein). In some embodiments, the cell comprising a target DNA is in vitro. Suitable nucleic acids comprising nucleotide sequences encoding a single unique gRNA and/or a RNA-guided nuclease (e.g., dCas9 protein) and/or a detectable molecule construct (e.g., a PUF domain linked to a fluorescent protein) include expression vectors, where the expression vectors may be recombinant expression vector.

In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc.

Suitable expression vectors include, but are not limited to, viral vectors (e.g. viral vectors based on vaccinia virus; poliovirus; adenovirus (see, e.g., Li et al., Invest Opthalmol. Vis. Sci., 35:2543-2549, 1994; Borras et al., Gene Ther., 6:515-524, 1999; Li and Davidson, Proc. Natl. Acad. Sci. USA, 92:7700-7704, 1995; Sakamoto et al., Hum. Gene Ther., 5:1088-1097, 1999; WO 94/12649, WO 93/03769; WO 93/19191; WO 94/28938; WO 95/11984 and WO 95/00655); adeno-associated virus (see, e.g., Ali et al., Hum. Gene Ther., 9:81-86, 1998, Flannery et al., Proc. Natl. Acad. Sci. USA, 94:6916-6921, 1997; Bennett et al., Invest Opthalmol Vis Sci 38:2857-2863, 1997; Jomary et al., Gene Ther., 4:683-690, 1997, Rolling et al., Hum. Gene Ther., 10:641-648, 1999; Ali et al., Hum. Mol. Genet., 5:591-594, 1996; Srivastava in WO 93/09239, Samulski et al., J. Vir., 63:3822-3828, 1989; Mendelson et al., Virol., 166: 154-165, 1988; and Flotte et al., Proc. Natl. Acad. Sci. USA, 90: 10613-10617, 1993); SV40; herpes simplex virus; human immunodeficiency virus (see, e.g., Miyoshi et al., Proc. Natl. Acad. Sci. USA, 94: 10319-23, 1997; Takahashi et al., J. Virol., 73:7812-7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, HIV virus, myeloproliferative sarcoma virus, and mammary tumor virus); and the like.

Numerous suitable expression vectors are known to those skilled in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al., Methods in Enzymology, 153:516-544, 1987).

### Kits

The present disclosure also provides a kit for carrying out a subject method. A subject kit may comprise: (a) a unique single gRNA of the present disclosure, or a nucleic acid (e.g., vector) comprising a nucleotide sequence encoding the same; optionally, (b) a subject catalytically-inactive RNA-guided nuclease (e.g., dCas9 protein), or a vector encoding the same (including an expressible mRNA encoding the same); and optionally, (c) one or more subject RBP domains (e.g., PUF domains) linked to detectable molecules, or a vector encoding the same (including an expressible mRNA encoding the same).

In some embodiments, one or more of (a)-(c) may be encoded by the same vector.

In some embodiments, the kit also comprises one or more buffers or reagents that facilitate the introduction of any one of (a)-(c) into a host cell, such as reagents for transformation, transfection, or infection.

For example, a subject kit can further include one or more additional reagents, where such additional reagents can be selected from: a buffer; a wash buffer; a control reagent; a control expression vector or RNA polynucleotide; a reagent for in vitro production of the RNA-guided nuclease (e.g., dCas9) or RBP domain construct from DNA; and the like.

Components of a subject kit can be in separate containers; or can be combined in a single container.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

### Example 1: One gRNA can be Used to Image One Non-Repetitive Locus

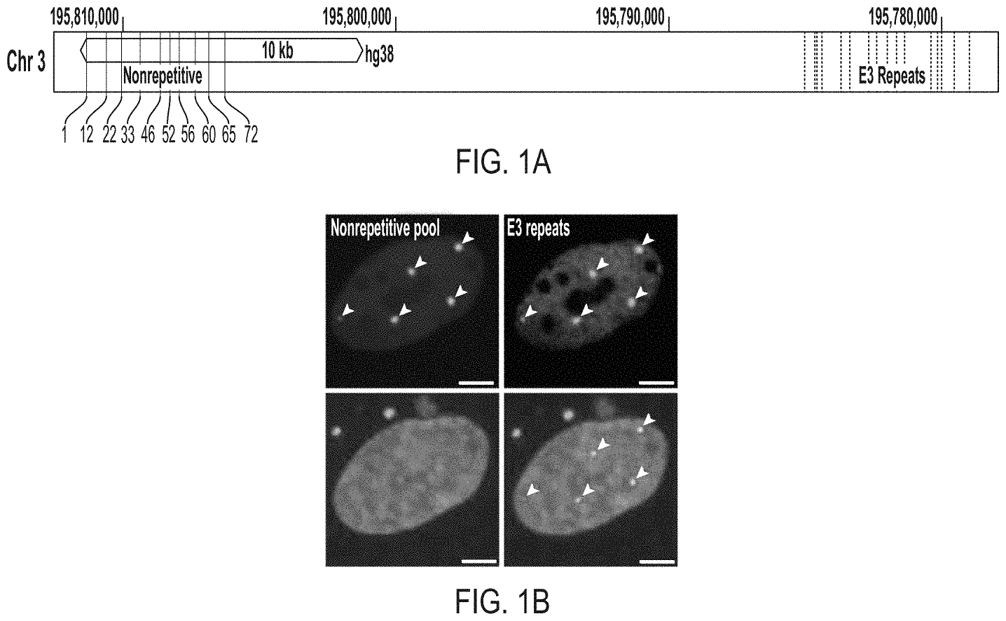

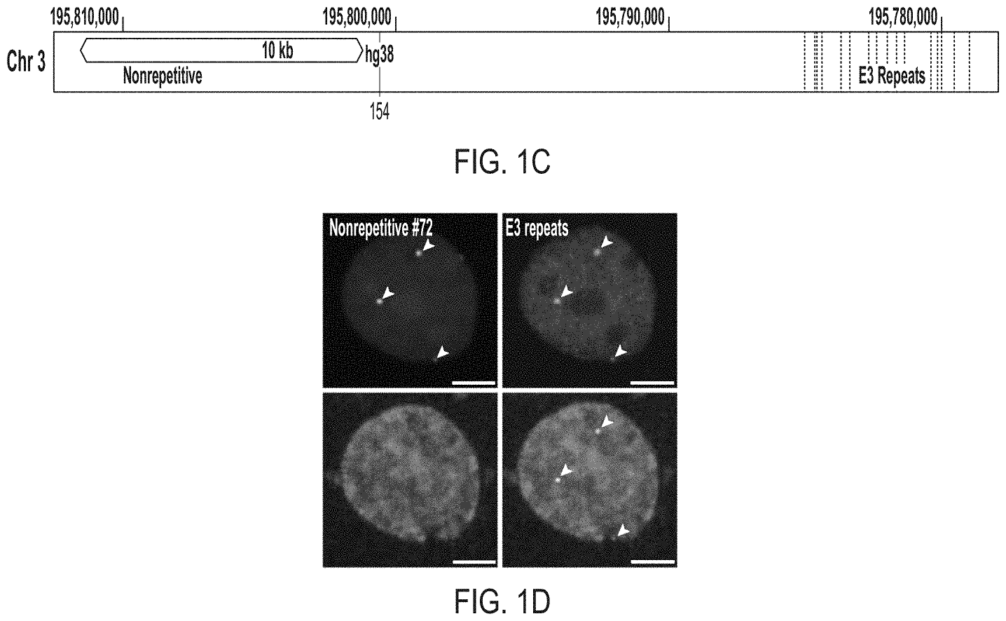

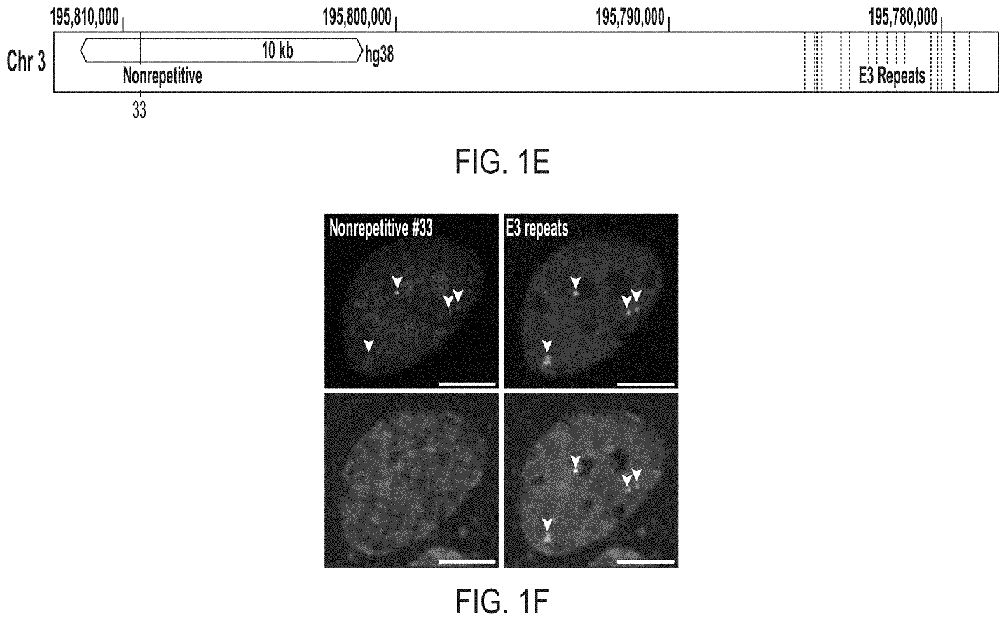

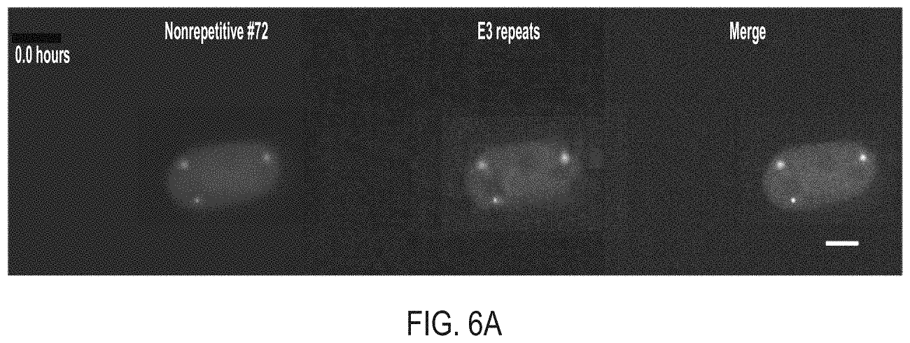

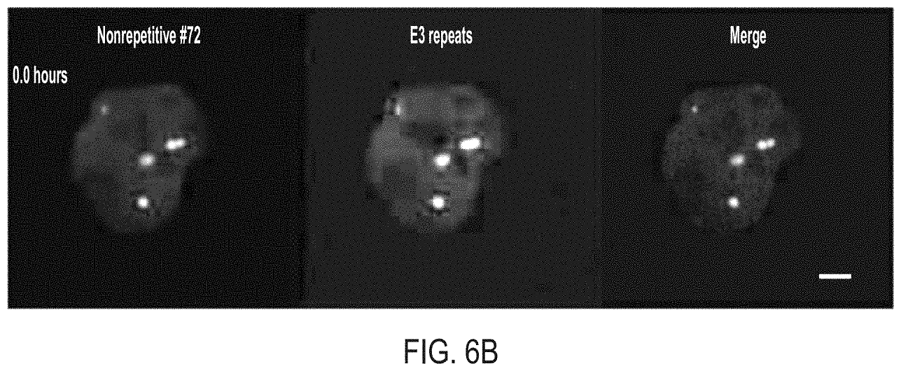

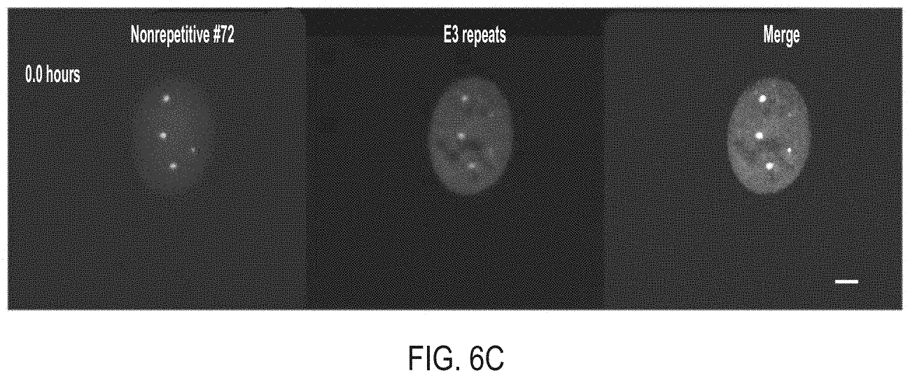

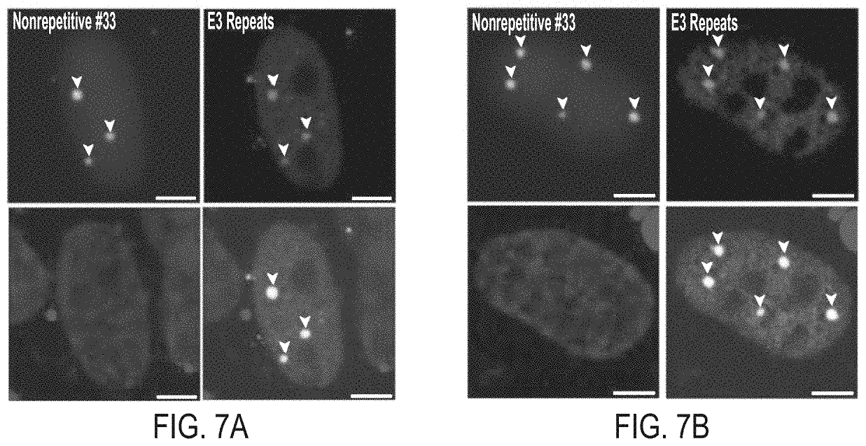

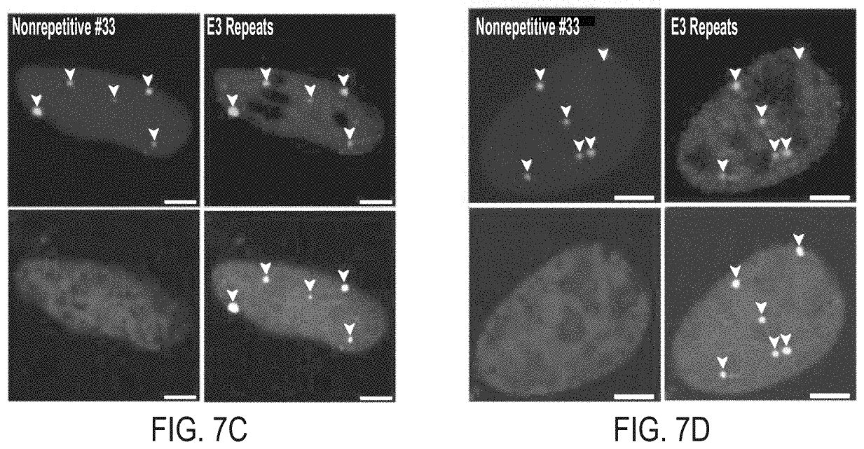

In order to show use of Casilio in imaging non-repetitive genomic loci, the MUC4 gene on chromosome 3 was targeted (FIG. 1A). Using a pool of ten gRNAs each targeting unique non-repetitive loci within a 5 kilobase (kb) region, co-labeling with guides to repetitive region E3 of exon 2 in human osteosarcoma U2OS cells was shown (FIG. 1B). Narrowing the number of gRNAs required to image non-repetitive loci to one gRNA, fluorescent foci at the single locus #72 was observed and confirmed by overlap with repetitive region E3 (FIGS. 1C-1D, FIG. 5). Time-lapse imaging revealed co-labeling up to 15 hours (FIG. 6). Additionally, single locus #33 co-labeled with repetitive region E3 is shown (FIGS. 1E-1F, FIG. 7).

### Example 2: Multiple Non-Repetitive Loci can be Labeled Using One gRNA for Each Locus

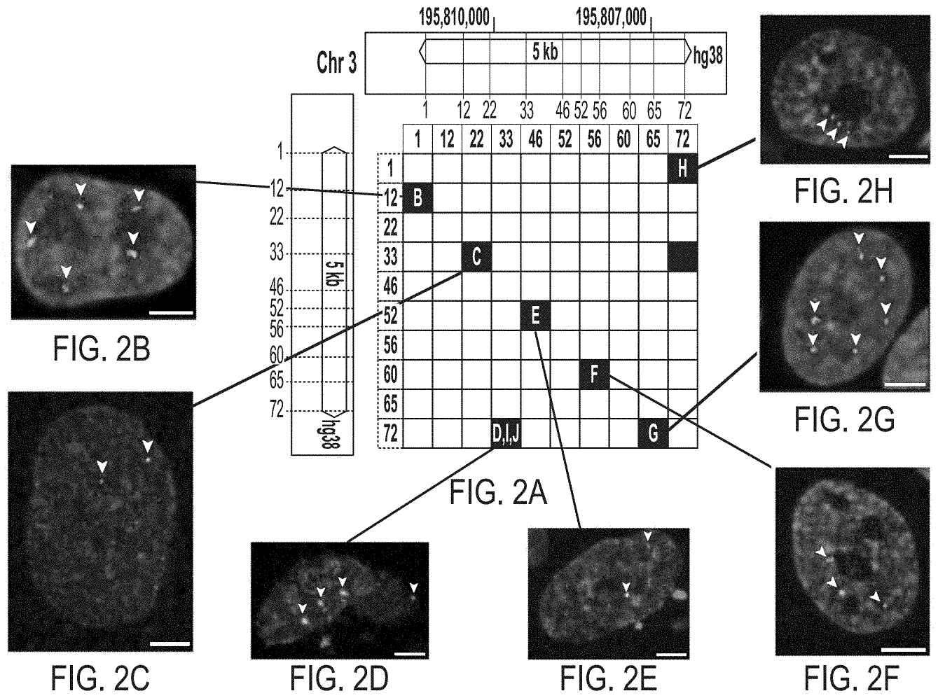

Testing whether two non-repetitive loci ≤5 kb apart could be simultaneously labelled by Casilio with two colors, one gRNA for each locus of the MUC4 gene was used (FIG. 2A). Eight pairs of non-repetitive loci showed co-labeling (FIGS. 2B-2H). Use of fluorescent proteins Clover and mRuby2 was interchangeable between the two targets. The dCas9 and the two non-repetitive gRNAs were expressed by plasmids. To show that Casilio labeling of non-repetitive loci can be applied to additional loci, the CISTR-ACT gene on chromosome 12 was targeted (FIG. 8A). Using one gRNA for each of two loci 1.2 kb apart in U2OS cells, co-labeling was demonstrated (FIG. 8B). The MUC4 (15) and CISTR-ACT (19) loci were previously only detected using gRNA pools.

### Example 3: Multiple Non-Repetitive Loci can be Labeled at an Increasing Distance from an Anchor Non-Repetitive Locus

For additional validation and to investigate the resolution of this approach, gRNAs were designed to target non-repetitive loci at increasing distances from non-repetitive single locus #33 on chromosome 3 (FIG. 3A). Measurements of loci pairs in images showed a separation of 0.14 μm on average for 8 kb from the anchor #33 (FIGS. 3B-3C). The average measured distance increased as the genomic target distance increased (FIGS. 3B, 3D-3J). Loci pairs showed an average separation of 0.16 μm for 14 kb, 0.29 μm for 19 kb, 0.51 μm for 24 kb, 1.19 μm for 28 kb, 1.21 μm for 44 kb, 1.66 μm for 58.5 kb, and 1.74 μm for 74 kb from the anchor #33 (FIGS. 3B, 3D-3J). This supports the earlier results described herein, in which co-labeling was seen with non-repetitive locus #72 and repetitive region E3 which are 21 kb apart at the closest point (FIGS. 1C-1D, FIG. 5). It additionally supports the earlier results described herein, in which co-labeling is seen with non-repetitive locus #33 and repetitive region E3 which are 24 kb apart at the closest point (FIGS. 1E-1F, FIG. 7). The distance per kb is consistent among each average pair: 0.018 μm/kb for 8 kb, 0.012 μm/kb for 14 kb, 0.015 μm/kb for 19 kb, 0.021 μm/kb for 24 kb, 0.043 μm/kb for 28 kb, 0.028 μm/kb for 44 kb, 0.028 μm/kb for 58.5 kb, and 0.024 μm/kb for 74 kb.

### Example 4: Casilio Live Cell Imaging of Chromatin Interactions

To test whether Casilio can be applied to study the temporal dynamics of chromatin interactions in live cells, we selected two chromatin interactions from published cohesin (RAD21) ChIA-PET dataset (ENCSR110JOO, Michael Snyder lab) with 50 kb and 362 kb genomic separation and designed a pair of one-copy gRNAs for each interaction pair using a combination of ChIA-PET2, JACKIE and Cas-OFFinder {Li, 2017 #24; Zhu, 2020 #25; Bae, 2014 #26} (FIGS. 9A-9G). Live cell microscopy of ARPE-19 cells transfected with dual-color Casilio probe pairs revealed highly dynamic chromatin interactions at second timescales (FIGS. 9B-9G), demonstrating the capabilities of Casilio to image pairwise interactions of nonrepetitive sequence elements with high spatial and temporal resolution.

### Example 5: Casilio Live Cell Imaging of Five Sequential Loci

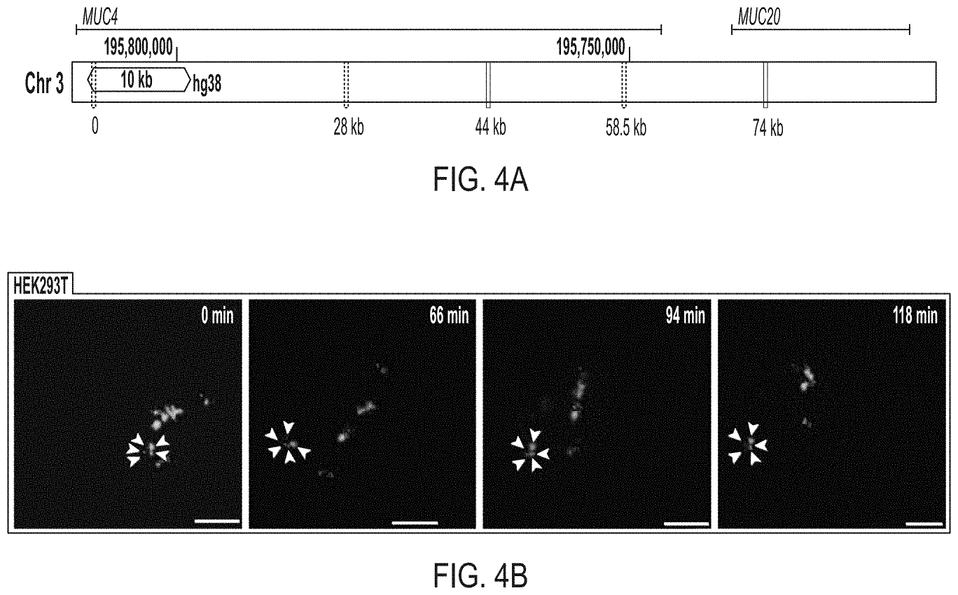

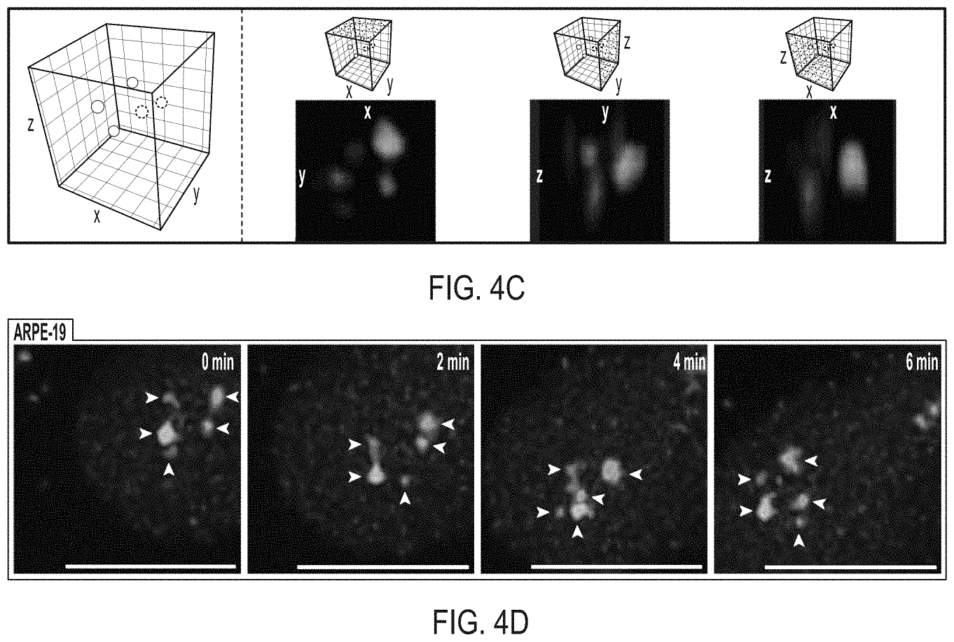

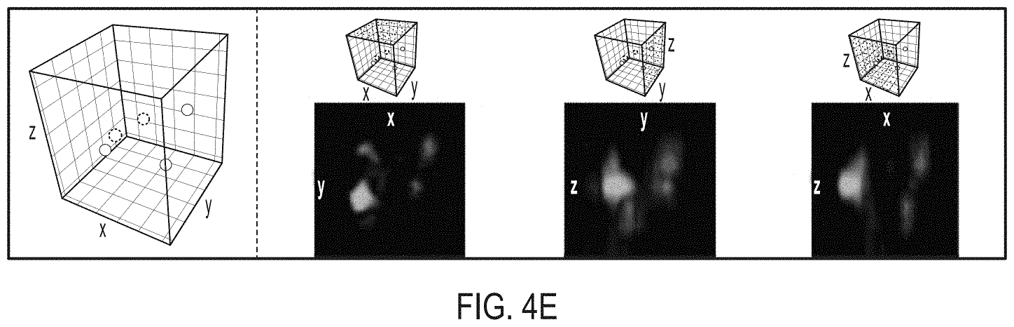

While imaging specific interactions of non-coding elements such as enhancers and promoters will provide much information about the gene regulation, visualizing a continuous stretch of genomic region will inform us about the structural folding dynamics and illuminate the process of chromatin loop formation. Given the low gRNA requirement of Casilio allowing imaging of each nonrepetitive locus with one sgRNA, we next explored the possibility of imaging multiple nonrepetitive loci simultaneously, specifically for tracking the structure of a continuous genomic region. We call this technique of deploying sequential Casilio probes across a stretch of genomic DNA “Programmable Imaging of Structure with Casilio Emitted Sequence of signal”—PISCES. To reduce the number of plasmids for transfection, we first constructed a plasmid with an array of five gRNAs targeting location 0, 28 kb, 44 kb, 58.5 kb, and 74 kb with alternating 15×PBSc or 15×PBS9R scaffolds (FIG. 4A). Five clustered fluorescent foci (three green plus two magenta) was observed in HEK293T cells (FIG. 4B and 4C) and ARPE-19 cells (FIGS. 4D and 4E) transfected with the pentameric gRNA arrays. These results demonstrate the use of a dual-color code to encode a sequence of Casilio imaging probes for illuminating the dynamic folding of a >70 kb genomic region in live cells.

In this study, we present a CRISPR/Cas-based method for live cell fluorescence imaging of nonrepetitive genomic loci with low gRNA requirements (1 gRNA/locus) and high spatiotemporal resolutions, allowing resolution of <28 kb at second timescales. We applied Casilio to visualize the dynamics of interactions of two pairs of cohesion-bound elements in native, unmodified chromosomes. Using a binary code of two fluorescent proteins (PISCES), we showed that folding of a continuous stretch of DNA can be imaged over time. These tools revealed highly heterogeneous and dynamic nature of chromatin folding and interactions, further supporting the need to study 4D nucleome with high spatiotemporal resolutions. The reduction of gRNA requirement compared with previously published CRISPR-based approaches will not only significantly reduce the technical challenge in applying live cell imaging to study chromatin interactions in hard-to-transfect cells, but also simplify future design of genome-wide imaging gRNA libraries.

### Materials and Methods

Cloning

Guide sequences were under control of human U6 promoter. They were cloned into gRNA-PBS expression vectors pAC1372-pX-gRNA-15×PBS a (Addgene #71889) or pAC1373-pX-gRNA-25×PBSa (Addgene #71890) or pAC1430-pX-gRNA-15×PBSc (Addgene #71930) via BbsI. dCas9 expression plasmid pAC1445-pmax-dCas9 was previously described (Addgene #73169). Clover and mRuby2 with PUF RNA-binding domain were produced using expression vectors pAC1446 (Clover_PUFa) (Addgene #73688), pAC1447 (Clover_PUFc) (Addgene #73689) and pAC1448 (mRuby2_PUFa) (Addgene #73690).

Cell Culture

Human osteosarcoma U2OS cells (ATCC® HTB-96TH) and human embryonic kidney HEK293T cells (ATCC® CRL3216™) were cultivated in Dulbecco's modified Eagle's medium (DMEM) (Sigma) with 10% fetal bovine serum (Lonza), 4% Glutamax (Gibco), 1% Sodium Pyruvate (Gibco) and penicillin-streptomycin (Gibco). Human retinal pigment epithelial ARPE-19 cells (ATCC® CRL-2302™) were cultivated in DMEM/F12 (Gibco) with 10% fetal bovine serum (Lonza), and 1% penicillin-streptomycin (Gibco). Incubator conditions were humidified 37° C. and 5% CO2. Cell lines expressing constitutive dCas9 was generated by transducing cells lentiviruses prepared from a lenti-dCas9-Blast plasmid, followed by Blast selection.

Transfection with Plasmid DNA

U2OS/dCas9 cells were seeded at density of 55,000-130,000 cells/compartment in 35 mm 4-compartment CELLview cell culture dish (Greiner Bio-One) 24 hours before transfection. Cells were transfected with 75-300 ng of sgRNA plasmid DNA containing 15 Pumilio Binding Sites (PBS), 10-25 ng of Clover-PUF fusion plasmid DNA, and 15-25 ng of mRuby2-PUF fusion plasmid using 0.5-1.2 μl Attractene (Qiagen) or 1 μl Lipofectamine 3000 (Invitrogen). Media was changed at 24 hours post-transfection.

HEK293T/dCas9 cells were seeded at density of 200,000-225,000 cells/compartment in 35 mm 4-compartment CELLview cell culture dish (Greiner Bio-One) 18-19 hours before transfection. Cells were transfected with 50-300 ng of sgRNA-15×PBS plasmid DNA, 5-10 ng of Clover-PUF fusion plasmid DNA, and 40-75 ng of mRuby2-PUF fusion plasmid DNA using 0.75 μl Lipofectamine 3000 (Invitrogen).

ARPE-19/dCas9 cells were seeded at density of 50,000-110,000 cells/compartment in 35 mm 4-compartment CELLview cell culture dish (Greiner Bio-One) 6-28 hours before transfection. Cells were transfected with 200-600 ng of sgRNA-15×PBS plasmid, and 5-40 ng of Clover-PUM fusion plasmid DNA, and 30-700 ng of PUF-mRuby2 fusion plasmid DNA using 1.5-1.7 μl Lipofectamine LTX (Invitrogen). Media was changed at 24 hours post-transfection.

Transfection with Plasmid DNA, dCas9 Protein, and IVT gRNA

Cells were seeded at density of 80,000-120,000 cells/compartment in 35 mm 4-compartment CELLview cell culture dish (Greiner Bio-One) the day before transfection. U2OS cells were transfected with 10-15 ng of PUF-fluorescent fusion plasmid DNA using 1 μl Lipofectamine 3000 (Invitrogen) Immediately after, cells were transfected with 500 ng Alt-R S.p. dCas9 protein V3 (IDT) and 130 ng gRNA containing 15 PUF-binding sites using Lipofectamine CRISPRMAX (Invitrogen).

Nuclear Staining

Prior to imaging, cells were stained with 0.5-1.0 μg/ml Hoechst prepared in cell culture media for 30-60 minutes, followed by two media washes.

Confocal Microscopy

Imaging was at 48-72 hours post-transfection. Images were acquired with the Dragonfly High Speed Confocal Platform 505 (Andor) using a Zyla sCMOS camera and a Leica HC PL APO 63x/1.47NA OIL CORR TIRF objective mounted on a Leica DMi8 inverted microscope equipped with a live-cell environmental chamber (Okolab) at humidified 37° C. and 5% CO2. Imaging mode was Confocal 40 μm. Hoechst images were acquired with a 200 mW solid state 405 nm laser and 450/50 nm BP emission filter. Clover images were acquired with a 150 mW solid state 488 nm laser and 525/50 nm BP emission filter. mRuby2 images were acquired with a 150 mW solid state 561 nm laser and 620/60 nm BP emission filter. Z-series covering the full nucleus was acquired at 0.13-1.0 μm step size. For time-lapse imaging, the Z-series was acquired at 0.3-4.1 μm step size. Images are maximum intensity projection of Z-series.

Image Processing

Raw 4D images of multiple non-repetitive sequential loci were processed using Fusion software robust (iterative) deconvolution algorithm with the presharpening filter at 50, denoising filter size 0.7, and 24 iterations.

Image Analysis

Imaris (Bitplane) image analysis software was used to measure spot distances. Z-series acquired at 0.19 μm or 0.5 μm step size was used. For each channel, spots were segmented based on maximum intensity in the 3D volume. Measurement points were set to intersect with the center of the spot object. With line mode set as pairs, distances between loci pairs in the 3D volume were measured from a spot in one channel to the closest spot in another channel.

### SEQUENCES

| | gRNA target | |
|---|---|---|
| Region | sequence | gRNA sequence |
| MUC4 E3 | GTGGCGTGACCTGTG | CAGCATCCACAGGTC |
| repeat | GATGCTG | ACGCCAC |
| | (SEQ ID NO: 1) | (SEQ ID NO: 22) |
| MUC4 non- | GAAGAGTGGAGGCCG | CCGCGCACGGCCTCC |
| repetitive #1 | TGCGCGG | ACTCTTC |
| | (SEQ ID NO: 2) | (SEQ ID NO: 23) |
| MUC4 non- | GCAAGCAAGGGAAGC | CCTTGTCGCTTCCCT |
| repetitive | GACAAGG | TGCTTGC |
| #12 | (SEQ ID NO: 3) | (SEQ ID NO: 24) |
| MUC4 non- | GATGTTTCAGGACTA | TCAGCCTAGTCCTGA |
| repetitive | GGCTGA | AACATC |
| #22 | (SEQ ID NO: 4) | (SEQ ID NO: 25) |
| MUC4 non- | GAGCTGGGCCAGGAG | TCTCCTCTCCTGGCC |
| repetitive | AGGAGA | CAGCTC |
| #33 | (SEQ ID NO: 5) | (SEQ ID NO: 26) |
| MUC4 non- | GAGGGGTCTGTGGAG | AAACTCTCCACAGAC |
| repetitive | AGTTT | CCCTC |
| #46 | (SEQ ID NO: 6) | (SEQ ID NO: 27) |
| MUC4 non- | GTGGAGACAGGGTTG | GCTTGGCCAACCCTG |
| repetitive | GCCAAGC | TCTCCAC |
| #52 | (SEQ ID NO: 7) | (SEQ ID NO: 28) |
| MUC4 non- | GGCTTGGTGTATTCA | CATTCTGAATACACC |
| repetitive | GAATG | AAGCC |
| #56 | (SEQ ID NO: 8) | (SEQ ID NO: 29) |
| MUC4 non- | GACAGAGTTTCTCTC | GGGGGACAGAGAGAA |
| repetitive | TGTCCCCC | ACTCTGTC |
| #60 | (SEQ ID NO: 9) | (SEQ ID NO: 30) |
| MUC4 non- | GTAGAGATGCCGCCC | GGGCGGGGCGGCATC |
| repetitive | CGCCC | TCTAC |
| #65 | (SEQ ID NO: 10) | (SEQ ID NO: 31) |
| MUC4 non- | GACAAGTCAGGAAGG | CACAGGGCCCTTCCT |
| repetitive | GCCCTGTG | GACTTGTC |
| #72 | (SEQ ID NO: 11) | (SEQ ID NO: 32) |
| 8 kb from | GCTCCCCAAGTTTAT | AACGCATAAACTTGG |
| non- | GCGTT | GGAGC |
| repetitive | (SEQ ID NO: 12) | (SEQ ID NO: 33) |
| #33 | | |
| 14 kb from | GGCGGTTCCAGCTTT | TGCAAAAAGCTGGAA |
| non- | TTGCA | CCGCC |
| repetitive | (SEQ ID NO: 13) | (SEQ ID NO: 34) |
| #33 | | |
| 19 kb from | GTTGATGTTGTAACC | TCACACCGGTTACAA |
| non- | GGTGTGA | CATCAAC |
| repetitive | (SEQ ID NO: 14) | (SEQ ID NO: 35) |
| #33 | | |
| 24 kb from | GCATCGGTGTCATGA | CCGCTCTTCATGACA |
| non- | AGAGCGG | CCGATGC |
| repetitive | (SEQ ID NO: 15) | (SEQ ID NO: 36) |
| #33 | | |
| 28 kb from | GCCGGTGACAGGAAG | GCACTCTTCCTGTCA |
| non- | AGTGC | CCGGC |
| repetitive | (SEQ ID NO: 16) | (SEQ ID NO: 37) |
| #33 | | |
| 44 kb from | GAATCCACCAAGAAG | GGGCCTCTTCTTGGT |
| non- | AGGCCC | GGATTC |
| repetitive | (SEQ ID NO: 17) | (SEQ ID NO: 38) |
| #33 | | |
| 58.5 kb | GAAGTCAATGACCGG | TCGGGGCCCGGTCAT |
| from non- | GCCCCGA | TGACTTC |
| repetitive | (SEQ ID NO: 18) | (SEQ ID NO: 39) |
| #33 | | |
| 74 kb from | GGATTGGAGCAAAGA | CACCACTTCTTTGCT |
| non- | AGTGGTG | CCAATCC |
| repetitive | (SEQ ID NO: 19) | (SEQ ID NO: 40) |
| #33 | | |
| 94 kb from | GACAGACTCATCCCC | GTCCTGGGGATGAGT |
| non- | AGGAC | CTGTC |
| repetitive | (SEQ ID NO: 51) | (SEQ ID NO: 52) |
| #33 | | |
| CISTR- | GGTCGTCAAGACCAA | CTTGGTTGGTCTTGA |
| ACT-1 | CCAAG | CGACC |
| | (SEQ ID NO: 20) | (SEQ ID NO: 41) |
| CISTR- | GACCTTGGAGGGGAG | TTGGGCTCCCCTCCA |
| ACT-4 | CCCAA | AGGTC |
| | (SEQ ID NO: 21) | (SEQ ID NO: 42) |
| FIG. 3B- | GGGTAAGAAGCCACT | ACCCTAGTGGCTTCT |
| D: Locus A | AGGGT | TACCC |
| near | (SEQ ID NO: 43) | (SEQ ID NO: 47) |
| MASP1 | | |
| FIG. 3B- | GCATAGCCGCATTTG | GCTTTCAAATGCGGC |
| D: Locus B | AAAGC | TATGC |
| near BCL6 | (SEQ ID NO: 44) | (SEQ ID NO: 48) |
| FIG. 3E- | GTGAATAAGACCAAC | AGAGGGTTGGTCTTA |
| G: Locus A | CCTCT | TTCAC |
| in CDC6 | (SEQ ID NO: 45) | (SEQ ID NO: 49) |
| FIG. 3E- | GCGGGTGATCACGCT | TCTTCAGCGTGATCA |
| G: Locus B | GAAGA | CCCGC |
| in RARA | (SEQ ID NO: 46) | (SEQ ID NO: 50) |

### REFERENCES

1. Bustin, M. and Misteli, T. Nongenetic functions of the genome. Science 352, 671-678, (2016).
2. Maass, P. G. et al. Interchromosomal interactions: A genomic love story of kissing chromosomes. J. Cell Biol. DOI: 10.1083/jcb.201806052 (2018).
3. Mishra, A. and Hawkins, R. D. Three-dimensional genome architecture and emerging technologies: looping in disease. Genome Med. 9, 87 (2017).
4. Krumm, A. and Duan, Z. Understanding the 3D genome: Emerging impacts on human disease. Semin. Cell Dev. Biol. DOI: 10.1016/j.semcdb.2018.07.004 (2018).
5. Li, G. et al. Extensive promoter-centered chromatin interactions provide a topological basis for transcription regulation. Cell 148, 84-98 (2012).
6. Rao, S. S. P. et al. A 3D map of the human genome at kilobase resolution reveals principles of chromatin looping. Cell 159, 1665-1680, (2014).
7. Tang, Z. et al. CTCF-mediated human 3D genome architecture reveals chromatin topology for transcription. Cell 163, 1611-27 (2015).
8. Konermann, S. et al. Optical control of mammalian endogenous transcription and epigenetic states. Nature 500, 472-476 (2013).
9. Morisaki, T. et al. Single-molecule analysis of transcription factor binding at transcription sites in live cells. Nat. Commun. 5, 4456 (2014).
10. Zhao, Z. W. et al. Spatial organization of RNA polymerase II inside a mammalian cell nucleus revealed by reflected light-sheet superresolution microscopy. Proc. Natl. Acad. Sci. USA 111, 681-686 (2014).
11. Chen, B. et al. Imaging specific genomic DNA in living cells. Ann. Rev. Biophys. 45, 1-23 (2016).
12. Ma, H. et al. Visualization of repetitive DNA sequences in human chromosomes with transcription activator-like effectors. Proc. Natl. Acad. Sci. USA 110, 21048-21053 (2013).
13. Ren, R. et al. Visualization of aging-associated chromatin alterations with an engineered TALE system. Cell Res 27, 483-504 (2017).
14. Knight, S. C. et al. Genomes in focus: development and applications of CRISPR-Cas9 imaging technologies. Angew. Chem. Int. Ed. Engl. 57, 4329-4337 (2018).
15. Chen, B. et al. Dynamic imaging of genomic loci in living human cells by an optimized CRISPR/Cas system. Cell 155, 1479-1491 (2013).
16. Tanenbaum, M. E. et al. A protein-tagging system for signal amplification in gene expression and fluorescence imaging. Cell 159, 635-646 (2014).
17. Ma, H. et al. Multiplexed labeling of genomic loci with dCas9 and engineered gRNAs using CRISPRainbow. Nat. Biotechnol. 34, 528-30 (2016).
18. Qin, P. et al. Live cell imaging of low- and non-repetitive chromosome loci using CRISPR-Cas9 Nat. Commun. 10.1038/ncomms14725 (2017).
19. Ma, H. et al. CRISPR-Sirius: RNA scaffolds for signal amplification in genome imaging. Nat. Meth. 15, 928-931 (2018).
20. Maass, P. S. et al. Spatiotemporal allele organization by allele-specific CRISPR live-cell imaging (SNP-CLING). Nat. Strict. Mol. Biol. 25, 176-184 (2018).
21. Cheng, A. W. et al. Casilio: a versatile CRISPR-Cas9-Pumilio hybrid for gene regulation and genomic labeling. Cell Res. 26, 254-257 (2016).

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method comprising:
(a) imaging a live cell that comprises:
a catalytically-inactive ribonucleic acid (RNA)-guided nuclease;
a non-repetitive genomic locus bound by a single unique guide RNA (gRNA), wherein the gRNA comprises (i) a deoxyribonucleic (DNA)-targeting sequence that is complementary to the non-repetitive genomic locus, (ii) a RNA-guided nuclease-binding sequence, and (iii) a Pumilio-FBF (PUF) domain-binding sequence, and
a detectable molecule linked to a PUF domain that binds to the PUF domain-binding sequence of the gRNA; and
(b) detecting in the live cell the detectable molecule of the PUF domain bound to the PUF domain-binding sequence of the gRNA.

2. A method comprising:
(a) imaging a live cell that comprises:
a catalytically-inactive ribonucleic acid (RNA)-guided nuclease;
multiple non-repetitive genomic loci, wherein each non-repetitive locus is bound by a single unique guide RNA (gRNA), wherein the gRNA comprises (i) a deoxyribonucleic (DNA)-targeting sequence that is complementary to one of the non-repetitive genomic loci, (ii) a RNA-guided nuclease-binding sequence, and (iii) a Pumilio-FBF (PUF) domain-binding sequence, and
a detectable molecule linked to a PUF domain that binds to the PUF domain-binding sequence of the gRNA; and
(b) co-detecting in the live cell at the multiple non-repetitive genomic loci the detectable molecule of the PUF domain bound to the PUF domain-binding sequence of the gRNAs.

3. A method comprising
(a) contacting a live cell with
a catalytically-inactive ribonucleic acid (RNA)-guided nuclease or a polynucleotide encoding a RNA-guided nuclease,
multiple guide RNAs (gRNAs), a polynucleotide encoding multiple gRNAs, or multiple polynucleotides encoding a gRNA, wherein each of the gRNAs comprises (i) a deoxyribonucleic (DNA)-targeting sequence that is complementary to a single non-repetitive genomic locus in the live cell, (ii) a RNA-guided nuclease-binding sequence, and (iii) a Pumilio-FBF (PUF) domain-binding sequence, and
a fluorescent protein linked to a PUF domain or a polynucleotide encoding fluorescent protein linked to a PUF domain that binds to the PUF domain-binding sequence of each of the gRNAs; and
(b) co-detecting in the live cell the fluorescent protein linked to a PUF domain bound to the PUF domain-binding sequence of the gRNAs.

4. A method for imaging chromatin architecture, comprising:
labeling in a live cell a first non-repetitive chromatin anchor locus with (a) a single unique guide RNA (gRNA), wherein the gRNA comprises (i) a deoxyribonucleic (DNA)-targeting sequence that is complementary to the non-repetitive genomic locus, (ii) a RNA-guided nuclease-binding sequence, and (iii) a Pumilio-FBF (PUF) domain-binding sequence, and (b) a detectable molecule linked to a PUF domain that binds to the PUF domain-binding sequence of the gRNA;
labeling in the live cell multiple additional non-repetitive chromatin loci, each loci labeled with (a) a single unique gRNA, wherein the gRNA comprises (i) a DNA-targeting sequence that is complementary to the non-repetitive genomic locus, (ii) a RNA-guided nuclease-binding sequence, and (iii) a PUF domain-binding sequence, and (b) a detectable molecule linked to a PUF domain that binds to the PUF domain-binding sequence of the gRNA, wherein the multiple additional non-repetitive loci are located at increasing distances from the anchor locus; and
imaging in the live cell over a period of time the detectable molecules, thereby imaging chromatin architecture in the live cell.

5. The method of any one of claims 2-4, wherein the distance between at least two of the non-repetitive genomic loci is 1 kb to 5 kb.

6. The method of any one of claims 2-4, wherein the distance between at least two of the non-repetitive genomic loci is 1 kb to 200 kb.

7. The method of any one of claims 2-4, wherein the distance between at least two of the non-repetitive genomic loci is 10 kb to 200 kb.

8. The method of any one of claims 2-4, wherein the distance between at least two of the non-repetitive genomic loci is at least 1 kb, at least 5 kb, at least 10 kb, or at least 20 kb.

9. The method of any one of the foregoing claims, wherein the co-detecting of step (b) comprises time-lapse imaging of the live cell.

10. The method of any one of the foregoing claims, wherein the detectable molecule is a fluorescent protein.

11. The method of any one of claims 2-10, wherein the live cell is contacted with at least two PUF domains, each linked to a different detectable molecule, optionally wherein the detectable molecules are fluorescent proteins with different emission wavelengths relative to each other.

12. The method of any one of claims 2-11, wherein the live cell comprises at least two gRNAs, wherein each of the gRNAs comprises (i) a DNA-targeting sequence that is complementary to only a single non-repetitive genomic locus in the live cell, (ii) a RNA-guided nuclease-binding sequence, and (iii) a PUF domain-binding sequence.

13. The method of any one of claims 2-12, wherein the live cell comprises at least five gRNAs, wherein each of the gRNAs comprises (i) a DNA-targeting sequence that is complementary to only a single non-repetitive genomic locus in the live cell, (ii) a RNA-guided nuclease-binding sequence, and (iii) a PUF domain-binding sequence.

14. The method of any one of the foregoing claims, wherein the live cells do not include a pool of gRNAs.

15. The method of any one of the foregoing claims, wherein the catalytically-inactive RNA-guided nuclease is a dCas9 nuclease.

16. The method of any one of the foregoing claims, wherein at least one of the gRNAs comprises at least one copy of the PUF domain-binding sequence.

17. The method of any one of the foregoing claims, wherein the non-repetitive genomic loci or locus comprises chromatin.

18. An in vitro composition, comprising a live cell that comprises:
a catalytically-inactive ribonucleic acid (RNA)-guided nuclease;
multiple non-repetitive genomic loci, wherein each non-repetitive locus is bound by a single unique guide RNA (gRNA), wherein the gRNA comprises (i) a deoxyribonucleic (DNA)-targeting sequence that is complementary to one of the non-repetitive genomic loci, (ii) a RNA-guided nuclease-binding sequence, and (iii) a Pumilio-FBF (PUF) domain-binding sequence, and
a detectable molecule linked to a PUF domain that binds to the PUF domain-binding sequence of the gRNA.

19. The composition of claim 18, wherein the distance between at least two of the non-repetitive genomic loci is 1 kb to 5 kb.

20. The composition of claim 18, wherein the distance between at least two of the non-repetitive genomic loci is 1 kb to 200 kb.

21. The composition of claim 18, wherein the distance between at least two of the non-repetitive genomic loci is 10 kb to 200 kb.

22. The composition of claim 18, wherein the distance between at least two of the non-repetitive genomic loci is at least 1 kb, at least 5 kb, at least 10 kb, or at least 20 kb.

23. The composition of any one of the foregoing claims, wherein the detectable molecule is a fluorescent protein.

24. The composition of any one of claims 2-10, wherein the live cell comprises at least two PUF domains, each linked to a different detectable molecule, optionally wherein the detectable molecules are fluorescent proteins with different emission wavelengths relative to each other.

25. The composition of any one of the foregoing claims, wherein the live cell comprises at least three gRNAs, wherein each of the gRNAs comprises (i) a DNA-targeting sequence that is complementary to only a single non-repetitive genomic locus in the live cell, (ii) a RNA-guided nuclease-binding sequence, and (iii) a PUF domain-binding sequence.

26. The composition of any one of the foregoing claims, wherein the live cell comprises at least five gRNAs, wherein each of the gRNAs comprises (i) a DNA-targeting sequence that is complementary to only a single non-repetitive genomic locus in the live cell, (ii) a RNA-guided nuclease-binding sequence, and (iii) a PUF domain-binding sequence.

27. The composition of any one of the foregoing claims, wherein the live cell does not include a pool of gRNAs.

28. The composition of any one of the foregoing claims, wherein the catalytically-inactive RNA-guided nuclease is a dCas9 nuclease.

29. The composition of any one of the foregoing claims, wherein at least one of the gRNAs comprises at least one copy of the PUF domain-binding sequence.

30. The composition of any one of the foregoing claims, wherein the non-repetitive genomic loci comprise chromatin.

31. A method, comprising:
(a) imaging multiple non-repetitive genomic loci in a live cell, wherein each non-repetitive genomic locus is bound by a single unique guide RNA (gRNA), wherein the gRNA comprises (i) a deoxyribonucleic (DNA)-targeting sequence that is complementary to the non-repetitive genomic locus, (ii) a RNA-guided nuclease-binding sequence, and (iii) a RNA-binding protein (RBP) domain-binding sequence, and a detectable molecule linked to a RBP domain that binds to the RBP domain-binding sequence of the gRNA; and
(b) detecting in the live cell the detectable molecule of the RBP domain bound to the RBP domain-binding sequence of the gRNA.

32. The method of claim 31, wherein the non-repetitive genomic locus is chromatin.

33. The method of claim 31 or 32, wherein the RNA-guided nuclease-binding sequence is bound to dCas9 nuclease.

34. The method of any one of claims 31-33, wherein the detectable molecule is a fluorescent protein.

35. A method for detecting a chromosomal rearrangement in a cell, comprising:
delivering to a live cell (a) a catalytically-inactive RNA-guided nuclease, (b) a first single unique gRNA that comprises a DNA-targeting sequence that is designed to bind adjacent to and upstream from a nuclease cleavage site, (c) a detectable molecule linked to a PUF domain that binds to the PUF domain-binding sequence of the first gRNA, (d) a second single unique gRNA that comprises a DNA-targeting sequence that is designed to bind adjacent to and downstream from a nuclease cleavage site, and (e) a detectable molecule linked to a PUF domain that binds to the PUF domain-binding sequence of the second gRNA, wherein each gRNA further comprises a RNA-guided nuclease-binding sequence and a PUF domain-binding sequence; and
imaging in the live cell the distance between the first gRNA and the second gRNA to determine the presence or absence of a chromosomal rearrangement.

36. The method of claim 35, wherein the chromosomal rearrangement is a translocation, an inversion, or a duplication.

37. A method for identifying a genetic abnormality in a cell, comprising:
delivering to a live cell (a) a catalytically-inactive RNA-guided nuclease, (b) a first single unique gRNA that comprises a DNA-targeting sequence that is designed to bind adjacent to and upstream from a genetic abnormality, (c) a detectable molecule linked to a PUF domain that binds to the PUF domain-binding sequence of the first gRNA, (d) a second single unique gRNA that comprises a DNA-targeting sequence that is designed to bind adjacent to and downstream from a genetic abnormality, and (e) a detectable molecule linked to a PUF domain that binds to the PUF domain-binding sequence of the second gRNA, wherein each gRNA further comprises a RNA-guided nuclease-binding sequence and a PUF domain-binding sequence; and
imaging in the live cell the distance between the first gRNA and the second gRNA to determine the presence or absence of a chromosomal rearrangement.

38. The method of claim 37, wherein the genetic abnormality is a chromosomal rearrangement.

39. The method of claim 38, wherein the chromosomal rearrangement is a translocation, an inversion, or a duplication.


SEQUENCE LISTING (53 sequences)

SEQ ID NO: 1 - DNA, 22 bp, Artificial Sequence
  Synthetic Polynucleotide
gtggcgtgacctgtggatgctg

SEQ ID NO: 2 - DNA, 22 bp, Artificial Sequence
  Synthetic Polynucleotide
gaagagtggaggccgtgcgcgg

SEQ ID NO: 3 - DNA, 22 bp, Artificial Sequence
  Synthetic Polynucleotide
gcaagcaagggaagcgacaagg

SEQ ID NO: 4 - DNA, 21 bp, Artificial Sequence
  Synthetic Polynucleotide
gatgtttcaggactaggctga

SEQ ID NO: 5 - DNA, 21 bp, Artificial Sequence
  Synthetic Polynucleotide
gagctgggccaggagaggaga

SEQ ID NO: 6 - DNA, 20 bp, Artificial Sequence
  Synthetic Polynucleotide
gaggggtctgtggagagttt

SEQ ID NO: 7 - DNA, 22 bp, Artificial Sequence
  Synthetic Polynucleotide
gtggagacagggttggccaagc

SEQ ID NO: 8 - DNA, 20 bp, Artificial Sequence
  Synthetic Polynucleotide
ggcttggtgtattcagaatg

SEQ ID NO: 9 - DNA, 23 bp, Artificial Sequence
  Synthetic Polynucleotide
gacagagtttctctctgtccccc

SEQ ID NO: 10 - DNA, 20 bp, Artificial Sequence
  Synthetic Polynucleotide
gtagagatgccgccccgccc

SEQ ID NO: 11 - DNA, 23 bp, Artificial Sequence
  Synthetic Polynucleotide
gacaagtcaggaagggccctgtg

SEQ ID NO: 12 - DNA, 20 bp, Artificial Sequence
  Synthetic Polynucleotide
gctccccaagtttatgcgtt

SEQ ID NO: 13 - DNA, 20 bp, Artificial Sequence
  Synthetic Polynucleotide
ggcggttccagctttttgca

SEQ ID NO: 14 - DNA, 22 bp, Artificial Sequence
  Synthetic Polynucleotide
gttgatgttgtaaccggtgtga

SEQ ID NO: 15 - DNA, 22 bp, Artificial Sequence
  Synthetic Polynucleotide
gcatcggtgtcatgaagagcgg

SEQ ID NO: 16 - DNA, 20 bp, Artificial Sequence
  Synthetic Polynucleotide
gccggtgacaggaagagtgc

SEQ ID NO: 17 - DNA, 21 bp, Artificial Sequence
  Synthetic Polynucleotide
gaatccaccaagaagaggccc

SEQ ID NO: 18 - DNA, 22 bp, Artificial Sequence
  Synthetic Polynucleotide
gaagtcaatgaccgggccccga

SEQ ID NO: 19 - DNA, 22 bp, Artificial Sequence
  Synthetic Polynucleotide
ggattggagcaaagaagtggtg

SEQ ID NO: 20 - DNA, 20 bp, Artificial Sequence
  Synthetic Polynucleotide
ggtcgtcaagaccaaccaag

SEQ ID NO: 21 - DNA, 20 bp, Artificial Sequence
  Synthetic Polynucleotide
gaccttggaggggagcccaa

SEQ ID NO: 22 - DNA, 22 bp, Artificial Sequence
  Synthetic Polynucleotide
cagcatccacaggtcacgccac

SEQ ID NO: 23 - DNA, 22 bp, Artificial Sequence
  Synthetic Polynucleotide
ccgcgcacggcctccactcttc

SEQ ID NO: 24 - DNA, 22 bp, Artificial Sequence
  Synthetic Polynucleotide
ccttgtcgcttcccttgcttgc

SEQ ID NO: 25 - DNA, 21 bp, Artificial Sequence
  Synthetic Polynucleotide
tcagcctagtcctgaaacatc

SEQ ID NO: 26 - DNA, 21 bp, Artificial Sequence
  Synthetic Polynucleotide
tctcctctcctggcccagctc

SEQ ID NO: 27 - DNA, 20 bp, Artificial Sequence
  Synthetic Polynucleotide
aaactctccacagacccctc

SEQ ID NO: 28 - DNA, 22 bp, Artificial Sequence
  Synthetic Polynucleotide
gcttggccaaccctgtctccac

SEQ ID NO: 29 - DNA, 20 bp, Artificial Sequence
  Synthetic Polynucleotide
cattctgaatacaccaagcc

SEQ ID NO: 30 - DNA, 23 bp, Artificial Sequence
  Synthetic Polynucleotide
gggggacagagagaaactctgtc

SEQ ID NO: 31 - DNA, 20 bp, Artificial Sequence
  Synthetic Polynucleotide
gggcggggcggcatctctac

SEQ ID NO: 32 - DNA, 23 bp, Artificial Sequence
  Synthetic Polynucleotide
cacagggcccttcctgacttgtc

SEQ ID NO: 33 - DNA, 20 bp, Artificial Sequence
  Synthetic Polynucleotide
aacgcataaacttggggagc

SEQ ID NO: 34 - DNA, 20 bp, Artificial Sequence
  Synthetic Polynucleotide
tgcaaaaagctggaaccgcc

SEQ ID NO: 35 - DNA, 22 bp, Artificial Sequence
  Synthetic Polynucleotide
tcacaccggttacaacatcaac

SEQ ID NO: 36 - DNA, 22 bp, Artificial Sequence
  Synthetic Polynucleotide
ccgctcttcatgacaccgatgc

SEQ ID NO: 37 - DNA, 20 bp, Artificial Sequence
  Synthetic Polynucleotide
gcactcttcctgtcaccggc

SEQ ID NO: 38 - DNA, 21 bp, Artificial Sequence
  Synthetic Polynucleotide
gggcctcttcttggtggattc

SEQ ID NO: 39 - DNA, 22 bp, Artificial Sequence
  Synthetic Polynucleotide
tcggggcccggtcattgacttc

SEQ ID NO: 40 - DNA, 22 bp, Artificial Sequence
  Synthetic Polynucleotide
caccacttctttgctccaatcc

SEQ ID NO: 41 - DNA, 20 bp, Artificial Sequence
  Synthetic Polynucleotide
cttggttggtcttgacgacc

SEQ ID NO: 42 - DNA, 20 bp, Artificial Sequence
  Synthetic Polynucleotide
ttgggctcccctccaaggtc

SEQ ID NO: 43 - DNA, 20 bp, Artificial Sequence
  Synthetic Polynucleotide
gggtaagaagccactagggt

SEQ ID NO: 44 - DNA, 20 bp, Artificial Sequence
  Synthetic Polynucleotide
gcatagccgcatttgaaagc

SEQ ID NO: 45 - DNA, 20 bp, Artificial Sequence
  Synthetic Polynucleotide
gtgaataagaccaaccctct

SEQ ID NO: 46 - DNA, 20 bp, Artificial Sequence
  Synthetic Polynucleotide
gcgggtgatcacgctgaaga

SEQ ID NO: 47 - DNA, 20 bp, Artificial Sequence
  Synthetic Polynucleotide
accctagtggcttcttaccc

SEQ ID NO: 48 - DNA, 20 bp, Artificial Sequence
  Synthetic Polynucleotide
gctttcaaatgcggctatgc

SEQ ID NO: 49 - DNA, 20 bp, Artificial Sequence
  Synthetic Polynucleotide
agagggttggtcttattcac

SEQ ID NO: 50 - DNA, 20 bp, Artificial Sequence
  Synthetic Polynucleotide
tcttcagcgtgatcacccgc

SEQ ID NO: 51 - DNA, 20 bp, Artificial Sequence
  Synthetic Polynucleotide
gacagactcatccccaggac

SEQ ID NO: 52 - DNA, 20 bp, Artificial Sequence
  Synthetic Polynucleotide
gtcctggggatgagtctgtc

SEQ ID NO: 53 - protein, 349 aa, Homo Sapiens
GRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLG